(19) United States
(12) Patent Application Publication
(10) Pub. No.: US 2003/0166622 A1
(43) Pub. Date: Sep. 4, 2003

(54) Vitamin D derivatives with acyloxy groups in the side chains, process for their production, and the use for the production of pharmaceutical agents

(75) Inventors: Andreas Steinmeyer, Berlin (DE); Ulrich Zuegel, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/292,908

(22) Filed: Nov. 13, 2002

Related U.S. Application Data
(60) Provisional application No. 60/331,386, filed on Nov. 15, 2001.

(30) Foreign Application Priority Data
Nov. 13, 2001 (DE) 10156596.8

(51) Int. Cl. A61K 31/59; C07D 233/54; C07D 213/46; C07C 401/00

(52) U.S. Cl. 514/167; 552/653; 548/335.1; 546/315

(57) ABSTRACT

The invention relates to vitamin D derivatives of general formula I,

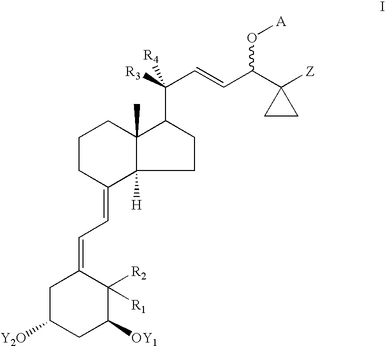

process for production, and the use for the production of pharmaceutical agents.

This application claims the benefit of the filing date of U.S. Provisional Application Serial No. 60/331,386 filed Nov. 15, 2001.

The invention relates to vitamin D derivatives of general formula I

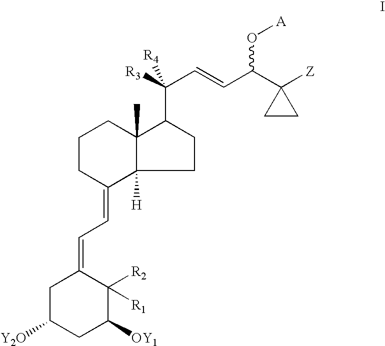

process for their production, as well as the use for the production of pharmaceutical agents.

Prior Art

Natural vitamins D₂ and D₃ are inherently biologically inactive and are converted into biologically active metabolites [1á,25-dihydroxy vitamin D₃ (calcitriol) or -D₂] only after hydroxylation at C-atom 25 in the liver and at C-atom 1 in the kidney. The action of the active metabolites involves the regulation of the calcium and phosphate concentration in the serum; they counteract a dropping of the calcium concentration in the serum by increasing the calcium absorption in the intestine and under certain circumstances promoting calcium mobilization from the bones. FIG. 1 shows the structure of some known vitamin D derivatives:

[image]

In addition to their pronounced effect on the calcium and phosphate metabolism, the active metabolites of vitamin D₂ and vitamin D₃ and their synthetic derivatives have a proliferation-inhibiting and differentiation-stimulating action on tumor cells and normal cells, such as, for example, skin cells. In addition, a pronounced effect on cells of the immune system (inhibiting of proliferation and interleukin-2-synthesis of lymphocytes, increase of cytotoxicity and phagocytosis in vitro of monocytes) was found, which manifests itself in an immunomodulatory action. Finally, because of a stimulating action on bone-forming cells, an increased formation of bone in normal and osteoporotic rats is found [R. Bouillon et al. “Short Term Course of 1,25-(OH)₂D₃ Stimulates Osteoblasts But Not Osteoclasts,” Calc. Tissue Int. 49, 168 (1991)].

All actions are mediated by binding to the vitamin D receptor. Because of the binding, the activity of specific genes is regulated.

When using biologically active metabolites of vitamins D₂ and D₃, a toxic effect on the calcium metabolism is produced (hypercalcemia).

By structural manipulations of the side chain, therapeutically usable effectiveness can be separated from undesirable hypercalcemic activity. A suitable structural variant is the introduction of a 24-hydroxy group. 1á-Cholecalciferols that are hydroxylated in 24-position are already described in DE 25 26 981. They have a lower toxicity than the corresponding non-hydroxylated 1á-cholecalciferol. Further, 24-hydroxy derivatives are described in the following patent applications: DE 39 33 034, DE 40 03 854, DE 40 34 730, EP 0 421 561, EP 0 441 467, WO 87/00834, and WO 91/12238.

Finally, 25-carboxylic acid derivatives of calcitriol that are hydroxylated at C-24 are described in WO 94/07853, and said derivatives exhibit a more advantageous spectrum of action than calcitriol. The equivalent is also true for new vitamin D derivatives with other substituents at C-25 (WO 97/00242). As the closest prior art for this invention, the applications WO 94/07853, WO 97/00242, and WO 97/41096 are considered.

While the ability to trigger a hypercalcemia is considerably weakened, proliferation-inhibiting and differentiation-stimulating actions are maintained. Generally, however, the introduction of the 24-hydroxyl group results in metabolic destabilization of the derivatives. For this reason, these compounds are only conditionally suitable for systemic administration. In particular, the bioavailability of the compounds is rather low owing to extensive hepatic catabolism.

There is therefore a need for new vitamin D derivatives that have as advantageous or improved a spectrum of action as the compounds that are described in the prior art (especially WO 94/07853 and WO 97/00242), but that are more advantageously suited for systemic, especially for oral, administration owing to their higher metabolic stability and thus improved oral bioavailability.

The object of this invention is therefore to make available such vitamin D derivatives. This object is achieved by the compounds that are disclosed in the claims.

It has been found, surprisingly enough, that the compounds that have acyloxy groups (especially substituted) at the 24-hydroxy group have in vivo biological activity, although they are inactive in vitro.

This invention therefore relates to vitamin D derivatives of general formula I,

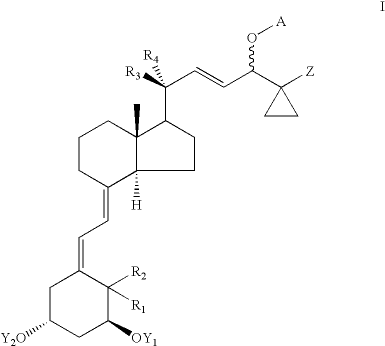

in which

R₁ and R₂ each mean a hydrogen atom or together an exocyclic methylene group,

R₃ and R₄, independently of one another, mean a hydrogen atom, a fluorine, chlorine or bromine atom, an alkyl group with 1 to 4 carbon atoms, together a methylene group or together with quaternary carbon atom 20 a 3- to 7-membered, saturated or unsaturated carbocyclic ring,

A means a group —C(X)—R⁵, —C(X)—NH—R⁵, —C(X)—N(R⁵)₂, —P(O)—(OR⁵)₂, —SO₂—OR⁵,

X stands for an oxygen atom or a sulfur atom,

R₅ means a straight-chain or branched, saturated or unsaturated alkyl chain with 1 to 10 carbon atoms, which optionally can be substituted by 1-3 hydroxy groups, a group COOR¹², a group CONR¹⁰R¹¹, a group P(O)(OR¹⁰)₂, P(O)(NR¹⁰R¹¹)₂, SO₃R¹⁰, S(O)₂NR¹⁰R¹¹, an N(R¹⁰R¹¹) group, whereby

R¹⁰ and R¹¹, independently of one another, mean a hydrogen atom or a straight-chain or branched, saturated or unsaturated alkyl chain with 1 to 10 carbon atoms, but R¹⁰ and R¹¹ cannot simultaneously mean hydrogen,

an aromatic or heteroaromatic radical with 5-12 carbon atoms, which optionally can be substituted with nitro groups, 1-2 optionally substituted C₁-C₆ alkyl groups, trihaloalkyl groups or alkoxy groups or halogen atoms, or an optionally substituted phenyl radical, benzyl radical or a 2-, 3- or 4-pyridyl radical,

Y₁ and Y₂, independently of one another, each mean a hydrogen atom or a group —C(O)R₆, whereby

R₆ stands for an aromatic or heteroaromatic radical with 5 to 12 C atoms, or for an aliphatic, straight-chain or branched, saturated or unsaturated C₁-C₁₂ alkyl radical, which optionally is interrupted by 1-2 oxygen atoms, 1-2 sulfur atoms and/or 1-2 NH groups and/or optionally is substituted by 1-2 hydroxy groups, 1-2 amino groups, 1-2 SH groups, 1-2 COOH groups and/or 1-2 phenyl groups,

Z means a straight-chain or branched, saturated or unsaturated 1-oxoalkyl group with 2 to up to 12 carbon atoms, a 1-oxo-(C₃-C₇)-cycloalkyl group, a benzoyl group, a 2-pyridylcarbonyl group, or a group CN, COOR₇, COSR₇, CONHR₇, CONR₇R₈, whereby

R₇ and R₈, independently of one another, stand for a hydrogen atom or a saturated or unsaturated alkyl group with 1 to 8 C atoms,

a cycloalkyl group with 3 to 8 carbon atoms,

a saturated or unsaturated alkyl group with 1 to 12 C atoms, which can have hydroxy or amino groups that are optionally substituted by A at any positions 1-3, halogen atoms or carboxylic acid esters or -amide units, and optionally is linked by a carbonyl group, a hydroxymethylene group or an ethenyldiyl unit (—CH═CH—, E- or Z-geometry) with carbon atom 25,

or a carbocyclic or heterocyclic optionally aromatic or heteroaromatic ring with 5- or 6-ring members, or a condensed ring system that consists of a 5- and a 6-membered ring or two 6-membered rings, which can be substituted by one or more halogen atoms, one or more hydroxy groups, one or more COOR₆ groups, 1-3 C₁-C₅ perfluoroalkyl groups, one or more C₁-C₅ alkyl groups, which in turn can be substituted by one or more halogen atoms, C₁-C₆ alkoxy groups and/or COOR₉ groups and/or can be interrupted by oxa, thia or aza functions, sulfoxy or sulfone groups, whereby

R₉ stands for a C₁-C₆ alkyl group, a benzyl group or a phenyl group, as well as all possible epimers or diastereomers and mixtures thereof.

In addition, this invention relates to the 24-hydroxy-calcitriol derivatives, which are disclosed in applications WO 87/00834, WO 94/07853, WO 97/00242, WO 97/41096, WO 99/16745, when their hydroxy group carries the above-defined radical A.

Preferred are vitamin D derivatives of formula I, in which

R₁ and R₂ each mean a hydrogen atom or together an exocyclic methylene group,

R₃ and R₄, independently of one another, mean a hydrogen atom, an alkyl group with 1 to 4 carbon atoms, together a methylene group or together with quaternary carbon atom 20 a 3-membered, saturated carbocyclic ring,

A means a group —C(X)—R⁵, —C(X)—NH—R⁵, or —C(X)—N(R⁵)₂,

X stands for an oxygen atom,

R₅ means a straight-chain or branched, saturated or unsaturated alkyl chain with 1 to 10 carbon atoms, which optionally can be substituted by 1-3 hydroxy groups, a group COOR¹², a group CONR¹⁰R¹¹, an N(R¹⁰R¹¹) group, whereby

R¹⁰ and R¹¹, independently of one another, mean a hydrogen atom or a straight-chain or branched, saturated or unsaturated alkyl chain with 1 to 10 carbon atoms, but R¹⁰ and R¹¹ cannot simultaneously mean hydrogen, an aromatic or heteroaromatic radical with 5-12 carbon atoms, which optionally can be substituted with nitro groups, 1-2 optionally substituted C₁-C₆ alkyl groups, trihaloalkyl groups or alkoxy groups or halogen atoms, or an optionally substituted phenyl radical, benzyl radical or a 2-, 3- or 4-pyridyl radical,

Y₁ and Y₂, independently of one another, each mean a hydrogen atom or a group —C(O)R₆, whereby

R₆ stands for an aromatic or heteroaromatic radical with 5 to 12 C atoms, or for an aliphatic, straight-chain or branched, saturated or unsaturated C₁-C₁₂ alkyl radical, which optionally is interrupted by 1-2 oxygen atoms, 1-2 sulfur atoms and/or 1-2 NH groups and/or optionally is substituted by 1-2 hydroxy groups, 1-2 amino groups, 1-2 SH groups, 1-2 COOH groups and/or 1-2 phenyl groups,

Z means a straight-chain or branched, saturated or unsaturated 1-oxoalkyl group with 2 to up to 12 carbon atoms, a 1-oxo-(C₃-C₇)-cycloalkyl group, a benzoyl group, a 2-pyridylcarbonyl group, or a group CN, COOR₇, COSR₇, CONHR₇, CONR₇R₈, whereby

R₇ and R₈, independently of one another, stand for a hydrogen atom or a saturated or unsaturated alkyl group with 1 to 8 C atoms,

a cycloalkyl group with 3 to 8 carbon atoms,

a saturated or unsaturated alkyl group with 1 to 12 C atoms, which can have hydroxy or amino groups that are optionally substituted by A at any positions 1-3, halogen atoms or carboxylic acid esters or -amide units, and optionally is linked by a carbonyl group, a hydroxymethylene group or an ethenyldiyl unit (—CH═CH—, E- or Z-geometry) with carbon atom 25,

or a carbocyclic or heterocyclic optionally aromatic or heteroaromatic ring with 5- or 6-ring members, or a condensed ring system that consists of a 5- and a 6-membered ring or two 6-membered rings, which can be substituted by one or more halogen atoms, one or more hydroxy groups, one or more COOR₆ groups, 1-3 C₁-C₅ perfluoroalkyl groups, one or more C₁-C₅ alkyl groups, which in turn can be substituted by one or more halogen atoms, C₁-C₆ alkoxy groups and/or COOR₉ groups and/or can be interrupted by oxa, thia or aza functions, sulfoxy or sulfone groups, whereby

R₉ stands for a C₁-C₆ alkyl group, a benzyl group or a phenyl group, as well as all possible epimers or diastereomers and mixtures thereof.

Another embodiment of the invention relates to vitamin D derivatives of formula I, in which

R₁ and R₂ each mean a hydrogen atom or together an exocyclic methylene group,

R₃ and R₄, independently of one another, mean a hydrogen atom, a methyl group with 1 to 4 carbon atoms, together a methylene group or together with quaternary carbon atom 20 a 3-membered, saturated carbocyclic ring,

A means a group —C(X)—R⁵, —C(X)—NH—R⁵, or —C(X)—N(R⁵)₂,

X stands for an oxygen atom,

R₅ means a branched, saturated or unsaturated alkyl chain with 3 to 10 carbon atoms, which optionally can be substituted by 1-3 hydroxy groups, an aromatic or heteroaromatic radical with 5-12 carbon atoms, which optionally can be substituted with 1-2 optionally substituted C₁-C₆ alkyl groups, or an optionally substituted phenyl radical, benzyl radical or a 2-, 3- or 4-pyridyl radical,

Y₁, and Y₂, independently of one another, each mean a hydrogen atom or a group —C(O)R₆, whereby

R₆ stands for an aromatic or heteroaromatic radical with 5 to 12 C atoms, or for an aliphatic, straight-chain or branched, saturated or unsaturated C₁-C₁₂ alkyl radical, which optionally is interrupted by 1-2 oxygen atoms, 1-2 sulfur atoms and/or 1-2 NH groups and/or optionally is substituted by 1-2 hydroxy groups, 1-2 amino groups, 1-2 SH groups, 1-2 COOH groups and/or 1-2 phenyl groups,

Z means a carbocyclic or heterocyclic, optionally aromatic or heteroaromatic ring with 5- or 6-ring members, or a condensed ring system that consists of a 5- and a 6-membered ring or two 6-membered rings, which can be substituted by one or more halogen atoms, one or more hydroxy groups, one or more COOR₆ groups, 1-3 C₁-C₅ perfluoroalkyl groups, one or more C₁-C₅ alkyl groups, which in turn can be substituted by one or more halogen atoms, C₁-C₆ alkoxy groups and/or COOR₉ groups and/or can be interrupted by oxa, thia or aza functions, sulfoxy or sulfone groups, whereby

R₉ stands for a C₁-C₆ alkyl group, a benzyl group or a phenyl group, as well as all possible epimers or diastereomers and mixtures thereof.

The invention also relates to a process for the production of the compounds according to the invention as well as the use of the compounds according to the invention for the production of pharmaceutical agents.

Especially advantageous embodiments of the invention are the subject of the subclaims or are clear owing to the preferences that are presented below.

The group —C(O)R₆, which is defined for Y₁ and Y₂, can carry 1 to 13 carbon atoms and is derived especially from saturated carboxylic acids and from benzoic acid and pyridinecarboxylic acid. The radicals can be cyclic, acyclic, straight-chain or branched, saturated or unsaturated, carbocyclic or heterocyclic. The radicals are preferably derived from C₁-C₉ carboxylic acids and from benzoic acid and pyridinecarboxylic acid. For example, formic acid, acetic acid, propionic acid, butanoic acid, pentanoic acid, pivalic acid, benzoic acid and 2- and 3- and 4-pyridinecarboxylic acid can be mentioned. The groups Y₁ and Y₂, independently of one another, especially preferably can each mean a hydrogen atom or a phenylcarbonyl group, pyridylcarbonyl group, acetyl group, propionyl group, butyryl group, isobutyryl group, valeryl group, isovaleryl group, hydroxyacetyl group, 1-hydroxypropionyl group, 2-hydroxypropionyl group, 3-hydroxypropionyl group, 1-hydroxybutyryl group, 2-hydroxybutyryl group, 3-hydroxybutyryl group or a 2,2-dimethyl-1-oxopropyl group.

Groups R₃ and R₄, independently of one another, can each mean a fluorine, chlorine or bromine atom, an alkyl group with 1 to 4 carbon atoms (methyl, ethyl, n-propyl, i-propyl, n-butyl, i-butyl, t-butyl), together a methylene group or together with quaternary carbon atom 20 a 3- to 7-membered, saturated or unsaturated carbocyclic ring.

For R₃ and R₄, the following preferred combinations apply: R₃=H, R₄=methyl or R₃=methyl, R₄=H; R₃=R₄=methyl; R₃ and R₄ together form a methylene group or together with tertiary carbon atom 20 form a cyclopropyl ring.

Alkyl groups R⁵ and R¹⁰ and R¹¹ can be straight-chain or branched, saturated or unsaturated and mean, e.g., methyl, ethyl, propyl, butyl, i-butyl, t-butyl, pentyl-i-pentyl, neopentyl, hexyl.

For R⁵, branched alkyl chains with 3 to 10 carbon atoms are preferred. Especially preferred are branched alkyl chains with 3 to 7 carbon atoms. Quite especially preferred are á-branched alkyl chains, such as, for example, 1-methylethyl, 1,1-dimethylethyl, 1-methylpropyl, 1,1-dimethylpropyl.

Alkyl group R⁵ can be substituted by one or two or three hydroxy groups. Preferred are substituted alkyl groups R⁵, which are substituted with one or two hydroxy groups. Especially preferred are substituted alkyl groups R⁵, which are substituted with a hydroxy group.

The benzyl group-and phenyl group R⁵ can be unsubstituted or else substituted by one or more halogen atom(s), hydroxy group(s), C₁-C₄ alkoxy group(s), CF₃ group(s) or amino group(s). The unsubstituted benzyl and phenyl groups as well as the pentafluorphenyl group are preferred.

Amino group R⁵ can carry at most one hydrogen atom. This disclaimer is to exclude more unstable carbamates.

R⁵ preferably means a straight-chain or branched, saturated or unsaturated alkyl chain with 1 to 10 carbon atoms, an aromatic or heteroaromatic radical with 5-12 carbon atoms, which optionally can be substituted with 1-2 optionally substituted C₁-C₆ alkyl groups, or an optionally substituted phenyl radical, benzyl radical or a 2-, 3- or 4-pyridyl radical.

R⁵ especially preferably means a branched, saturated or unsaturated alkyl chain with 3 to 10 carbon atoms, which optionally can be substituted by 1-3 hydroxy groups, an aromatic or heteroaromatic radical with 5-12 carbon atoms, which optionally can be substituted with 1-2 optionally substituted C₁-C₆ alkyl groups, or R⁵ means an optionally substituted phenyl radical or a 2-, 3- or 4-pyridyl radical. In particular, R⁵ can mean a branched, saturated or unsaturated alkyl chain with 3 to 10 carbon atoms, which optionally can be substituted by 1-3 hydroxy groups.

Z is preferably to mean a phenyl ring, which is substituted in ortho-, meta- or para-position with one or more methoxy, ethoxy, propoxy, hydroxy, fluorine, chlorine, bromine, methyl, ethyl, propyl, butyl, pentyl or trifluoromethyl groups, or Z is preferably to mean a heterocyclic system, such as, e.g., a furan, thiophene, pyrazole, pyrrole, oxazole, thiazole, imidazole ring, which can carry methyl, ethyl, propyl, isopropyl, tert-butyl, or pentyl groups at one or more of any position(s), and said groups in turn can be substituted by halogen of the hydroxy groups, and can be linked with the initial system via any C-atom, or Z is preferably to mean a heterocyclic condensed system, such as, e.g., a benzofuran, benzothiophene, benzimidazole, benzoxazole, benzothiazole, indole system, which can carry methyl, ethyl or propyl groups at any position, and said groups in turn can be substituted by halogen of the hydroxy groups, and can be linked with the initial system via any C-atom. Likewise, Z preferably is to be a straight-chain or branched 1-oxyalkyl chain with up to 8 carbon atoms or a carboxylic acid ester or a carboxylic acid amide with up to 8 carbons in the ester or amide chain.

Especially preferred for Z is a furan, thiophene, pyrazole, pyrrole, oxazole, thiazole, imidazole, 1,2,4-oxadiazole, benzofuran, benzothiophene, benzoxazole, benzothiazole, indole or benzimidazole ring that is substituted by a C₁-C₅ alkyl group. The above-mentioned alkyl group can be unsubstituted or substituted by one or two hydroxy groups.

If the term halogen is used, fluorine, chlorine, bromine or iodine in connection with substitution patterns of radicals, and preferably bromine or iodine, thus is meant as a leaving group in the process.

Of the compounds of general formula I according to the invention, the following compounds are quite especially preferred:

(5Z,7E)-(1S,3R,24S)-24-Acetoxy-25-(5-butyloxazole-2-yl)-26,27-cyclo-9,10-secocholesta-5,7,10(19)-triene-1,3-diol

(5Z,7E)-(1S,3R,24R)-24-acetoxy-25-(5-butyloxazole-2-yl)-26,27-cyclo-9,10-secocholesta-5,7,10(19)-triene-1,3-diol

(5Z,7E)-(1S,3R,24S)-25-(5-butyloxazole-2-yl)-24-(2,2-dimethyl-1-oxopropoxy)-26,27-cyclo-9,10-secocholesta-5,7,10(19)-triene-1,3-diol

(5Z,7E)-(1S,3R,24R)-25-(5-butyloxazole-2-yl)-24-(2,2-dimethyl-1-oxopropoxy)-26,27-cyclo-9,10-secocholesta-5,7,10(19)-triene-1,3-diol

(5Z,7E)-(1S,3R,24S)-25-(5-butyloxazole-2-yl)-24-(1-oxobutoxy)-26,27-cyclo-9,10-secocholesta-5,7,10(19)-triene-1,3-diol

(5Z,7E)-(1S,3R,24R)-25-(5-butyloxazole-2-yl)-24-(1-oxobutoxy)-26,27-cyclo-9,10-secocholesta-5,7,10(19)-triene-1,3-diol

(5Z,7E)-(1S,3R,24S)-25-(5-butyloxazole-2-yl)-24-(2-methyl-1-oxobutoxy)-26,27-cyclo-9,10-secocholesta-5,7,10(19)-triene-1,3-diol

(5Z,7E)-(1S,3R,24R)-25-(5-butyloxazole-2-yl)-24-(2-methyl-1-oxobutoxy)-26,27-cyclo-9,10-secocholesta-5,7,10(19)-triene-1,3-diol

(5Z,7E)-(1S,3R,24S)-25-(5-butyloxazole-2-yl)-24-(2-methyl-1-oxopropoxy)-26,27-cyclo-9,10-secocholesta-5,7,10(19)-triene-1,3-diol

(5Z,7E)-(1S,3R,24R)-25-(5-butyloxazole-2-yl)-24-(2-methyl-1-oxopropoxy)-26,27-cyclo-9,10-secocholesta-5,7,10(19)-triene-1,3-diol

(5Z,7E)-(1S,3R,24S)-25-(5-butyloxazole-2-yl)-24-(1-oxopropoxy)-26,27-cyclo-9,10-secocholesta-5,7,10(19)-triene-1,3-diol

(5Z,7E)-(1S,3R,24R)-25-(5-butyloxazole-2-yl)-24-(1-oxopropoxy)-26,27-cyclo-9,10-secocholesta-5,7,10(19)-triene-1,3-diol

(5Z,7E)-(1S,3R,24S)-25-(5-butyloxazole-2-yl)-24-(3-hydroxy-2-methyl-1-oxopropoxy)-26,27-cyclo-9,10-secocholesta-5,7,10(19)-triene-1,3-diol

(5Z,7E)-(1S,3R,24R)-25-(5-butyloxazole-2-yl)-24-(3-hydroxy-2-methyl-1-oxopropoxy)-26,27-cyclo-9,10-secocholesta-5,7,10(19)-triene-1,3-diol

(5Z,7E)-(1S,3R,24S)-25-(5-butyloxazole-2-yl)-24-(1-oxopentoxy)-26,27-cyclo-9,10-secocholesta-5,7,10(19)-triene-1,3-diol

(5Z,7E)-(1S,3R,24R)-25-(5-butyloxazole-2-yl)-24-(1-oxopentoxy)-26,27-cyclo-9,10-secocholesta-5,7,10(19)-triene-1,3-diol

(5Z,7E)-(1S,3R,24S)-25-(5-butyloxazole-2-yl)-26,27-cyclo-24-(4-pyridylcarbonyl)-9,10-secocholesta-5,7,10(19)-triene-1,3-diol

(5Z,7E)-(1S,3R,24R)-25-(5-butyloxazole-2-yl)-26,27-cyclo-24-(4-pyridylcarbonyl)-9,10-secocholesta-5,7,10(19)-triene-1,3-diol

(5Z,7E)-(1S,3R,24S)-25-(5-butyloxazole-2-yl)-24-[[(ethylamino)carbonyl]oxy]-26,27-cyclo-9,10-secocholesta-5,7,10(19)-triene-1,3-diol

(5Z,7E)-(1S,3R,24R)-25-(5-butyloxazole-2-yl)-24-[[(ethylamino)carbonyl]oxy]-26,27-cyclo-9,10-secocholesta-5,7,10(19)-triene-1,3-diol

(5Z,7E)-(1S,3R,24S)-25-(5-butyloxazole-2-yl)-24-[[(methylamino)carbonyl]oxy]-26,27-9,10-secocholesta-5,7,10(19)-triene-1,3-diol

(5Z,7E)-(1S,3R,24R)-25-(5-butyloxazole-2-yl)-24-[[(methylamino)carbonyl]oxy]-26,27-cyclo-9,10-secocholesta-5,7,10(19)-triene-1,3-diol

(5Z,7E)-(1S,3R,24S)-25-(5-butyloxazole-2-yl)-24-[[((pentafluorophenyl)amino)-carbonyl]oxy]-26,27-cyclo-9,10-secocholesta-5,7,10(19)-triene-1,3-diol

(5Z,7E)-(1S,3R,24R)-25-(5-butyloxazole-2-yl)-24-[[((pentafluorophenyl)amino)-carbonyl]oxy]-26,27-cyclo-9,10-secocholesta-5,7,10(19)-triene-1,3-diol

(5Z,7E)-(1S,3R,24S)-25-(5-butyloxazole-2-yl)-24-[[(2,2-dimethyl-3-hydroxy)-1-oxopropyl]oxy]-26,27-cyclo-9,10-secocholesta-5,7,10(19)-triene-1,3-diol

(5Z,7E)-(1S,3R,24R)-25-(5-butyloxazole-2-yl)-24-[[(2,2-dimethyl-3-hydroxy-1-oxopropyl]oxy]-26,27-cyclo-9,10-secocholesta-5,7,10(19)-triene-1,3-diol

(5Z,7E,22E)-(1S,3R,24S)-25-(5-butyloxazole-2-yl)-26,27-cyclo-9,10-secocholesta-5,7,10(19),22-tetraene-1,3,24-triol tris-isonicotinate

(5Z,7E,22E)-(1S,3R,24R)-25-(5-butyloxazole-2-yl)-26,27-cyclo-9,10-secocholesta-5,7,10(19),22-tetraene-1,3,24-triol tris-isonicotinate

(5Z,7E,22E)-(1S,3R,24S)-25-(5-butyloxazole-2-yl)-26,27-cyclo-9,10-secocholesta-5,7,10(19),22-tetraene-1,3,24-triol tribenzoate

(5Z,7E,22E)-(1S,3R,24R)-25-(5-butyloxazole-2-yl)-26,27-cyclo-9,10-secocholesta-5,7,10(19),22-tetraene-1,3,24-triol tribenzoate

(5Z,7E,22E)-(1S,3R,24S)-24-acetoxy-26,27-cyclo-9,10-secocholesta-5,7,10(19),22-tetraene-1,3-diol

(5Z,7E,22E)-(1S,3R,24R)-24-acetoxy-26,27-cyclo-9,10-secocholesta-5,7,10(19),22-tetraene-1,3-diol

(5Z,7E,22E)-(1S,3R,24S)-24-(2,2-dimethyl-1-oxopropoxy)-26,27-cyclo-9,10-secocholesta-5,7,10(19),22-tetraene-1,3-diol

(5Z,7E,22E)-(1S,3R,24R)-24-(2,2-dimethyl-1-oxopropoxy)-26,27-cyclo-9,10-secocholesta-5,7,10(19),22-tetraene-1,3-diol

(5Z,7E,22E)-(1S,3R,24S)-24-(1-oxobutoxy)-26,27-cyclo-9,10-secocholesta-5,7,10(19),22-tetraene-1,3-diol

(5Z,7E,22E)-(1S,3R,24R)-24-(1-oxobutoxy)-26,27-cyclo-9,10-secocholesta-5,7,10(19),22-tetraene-1,3-diol

(5Z,7E,22E)-(1S,3R,24S)-24-(2-methyl-1-oxobutoxy)-26,27-cyclo-9,10-secocholesta-5,7,10(19),22-tetraene-1,3-diol

(5Z,7E,22E)-(1S,3R,24R)-24-(2-methyl-1-oxobutoxy)-26,27-cyclo-9,10-secocholesta-5,7,10(19),22-tetraene-1,3-diol

(5Z,7E,22E)-(1S,3R,24S)-24-(1-oxopropoxy)-26,27-cyclo-9,10-secocholesta-5,7,10(19),22-tetraene-1,3-diol

(5Z,7E,22E)-(1S,3R,24R)-24-(1-oxopropoxy)-26,27-cyclo-9,10-secocholesta-5,7,10(19),22-tetraene-1,3-diol

(5Z,7E,22E)-(1S,3R,24S)-24-(1-oxopentoxy)-26,27-cyclo-9,10-secocholesta-5,7,10(19),22-tetraene-1,3-diol

(5Z,7E,22E)-(1S,3R,24R)-24-(1-oxopentoxy)-26,27-cyclo-9,10-secocholesta-5,7,10(19),22-tetraene-1,3-diol

(5Z,7E,22E)-(1S,3R,24S)-24-benzoyloxy-26,27-cyclo-9,10-secocholesta-5,7,10(19),22-tetraene-1,3-diol

(5Z,7E,22E)-(1S,3R,24R)-24-benzoyloxy-26,27-cyclo-9,10-secocholesta-5,7,10(19),22-tetraene-1,3-diol

(5Z,7E,22E)-(1S,3R,24S)-24-[[(methylamino)carbonyl]oxy]-26,27-cyclo-9,10-secocholesta-5,7,10(19),22-tetraene-1,3-diol

(5Z,7E,22E)-(1S,3R,24R)-24-[[(methylamino)carbonyl]oxy]-26,27-cyclo-9,10-secocholesta-5,7,10(19),22-tetraene-1,3-diol

(5Z,7E,22E)-(1S,3R,24S)-24-[[(ethylamino)carbonyl]oxy]-26,27-cyclo-9,10-secocholesta-5,7,10(19),22-tetraene-1,3-diol

(5Z,7E,22E)-(1S,3R,24R)-24-[[(ethylamino)carbonyl]oxy]-26,27-cyclo-9,10-secocholesta-5,7,10(19),22-tetraene-1,3-diol

(5Z,7E,22E)-(1S,3R,24S)-24-(4-pyridylcarbonyl)-26,27-cyclo-9,10-secocholesta-5,7,10(19),22-tetraene-1,3-diol

(5Z,7E,22E)-(1S,3R,24R)-24-(4-pyridylcarbonyl)-26,27-cyclo-9,10-secocholesta-5,7,10(19),22-tetraene-1,3-diol

(5Z,7E,22E)-(1S,3R,24S)-24-(3-hydroxy-2-methyl-1-oxopropoxy)-26,27-cyclo-9,10-secocholesta-5,7,10(19),22-tetraene-1,3-diol

(5Z,7E,22E)-(1S,3R,24R)-24-(3-hydroxy-2-methyl-1-oxopropoxy)-26,27-cyclo-9,10-secocholesta-5,7,10(19),22-tetraene-1,3-diol

(5Z,7E,22E)-(1S,3R,24S)-24-(2-methyl-1-oxopropoxy)-26,27-cyclo-9,10-secocholesta-5,7,10(19),22-tetraene-1,3-diol

(5Z,7E,22E)-(1S,3R,24R)-24-(2-methyl-1-oxopropoxy)-26,27-cyclo-9,10-secocholesta-5,7,10(19),22-tetraene-1,3-diol

(5Z,7E)-(1S,3R,24S)-24-[[((pentafluorophenyl)amino)-carbonyl]oxy]-26,27-cyclo-9,10-secocholesta-5,7,10(19)-triene-1,3-diol

(5Z,7E)-(1S,3R,24R)-24-[[((pentafluorophenyl)amino)-carbonyl]oxy]-26,27-cyclo-9,10-secocholesta-5,7,10(19)-triene-1,3-diol

(5Z,7E)-(1S,3R,24S)-24-[[(2,2-dimethyl-3-hydroxy)-1-oxopropyl]oxy]-26,27-cyclo-9,10-secocholesta-5,7,10(19)-triene-1,3-diol

(5Z,7E)-(1S,3R,24R)-24-[[(2,2-dimethyl-3-hydroxy)-1-oxopropyl]oxy]-26,27-cyclo-9,10-secocholesta-5,7,10(19)-triene-1,3-diol

(5Z,7E,22E)-(1S,3R,24S)-26,27-cyclo-9,10-secocholesta-5,7,10(19),22-tetraene triol tris-isonicotinate

(5Z,7E,22E)-(1S,3R,24R)-26,27-cyclo-9,10-secocholesta-5,7,10(19),22-tetraene-1,3,24-triol tris-isonicotinate

(5Z,7E,22E)-(1S,3R,24S)-26,27-cyclo-9,10-secocholesta-5,7,10(19),22-tetraene-1,3,24-triol tribenzoate

(5Z,7E,22E)-(1S,3R,24R)-26,27-cyclo-9,10-secocholesta-5,7,10(19),22-tetraene-1,3,24-triol tribenzoate

(5Z,7E)-(1S,3R,24S)-24-acetoxy-25-(5-butylthiazole-2-yl)-26,27-cyclo-9,10-secocholesta-5,7,10(19)-triene-1,3-diol

(5Z,7E)-(1S,3R,24R)-24-acetoxy-25-(5-butylthiazole-2-yl)-26,27-cyclo-9,10-secocholesta-5,7,10(19)-triene-1,3-diol

(5Z,7E)-(1S,3R,24S)-25-(5-butylthiazole-2-yl)-24-(2,2-dimethyl-1-oxopropoxy)-26,27-cyclo-9,10-secocholesta-5,7,10(19)-triene-1,3-diol

(5Z,7E)-(1S,3R,24R)-25-(5-butylthiazole-2-yl)-24-(2,2-dimethyl-1-oxopropoxy)-26,27-cyclo-9,10-secocholesta-5,7,10(19)-triene-1,3-diol

(5Z,7E)-(1S,3R,24S)-25-(5-butylthiazole-2-yl)-24-(1-oxobutoxy)-26,27-cyclo-9,10-secocholesta-5,7,10(19)-triene-1,3-diol

(5Z,7E)-(1S,3R,24R)-25-(5-butylthiazole-2-yl)-24-(1-oxobutoxy)-26,27-cyclo-9,10-secocholesta-5,7,10(19)-triene-1,3-diol

(5Z,7E)-(1S,3R,24S)-25-(5-butylthiazole-2-yl)-24-(2-methyl-1-oxobutoxy)-26,27-cyclo-9,10-secocholesta-5,7,10(19)-triene-1,3-diol

(5Z,7E)-(1S,3R,24R)-25-(5-butylthiazole-2-yl)-24-(2-methyl-1-oxobutoxy)-26,27-cyclo-9,10-secocholesta-5,7,10(19)-triene-1,3-diol

(5Z,7E)-(1S,3R,24S)-25-(5-butylthiazole-2-yl)-24-(1-oxopropoxy)-26,27-cyclo-9,10-secocholesta-5,7,10(19)-triene-1,3-diol

(5Z,7E)-(1S,3R,24R)-25-(5-butylthiazole-2-yl)-24-(1-oxopropoxy)-26,27-cyclo-9,10-secocholesta-5,7,10(19)-triene-1,3-diol

(5Z,7E)-(1S,3R,24S)-25-(5-butylthiazole-2-yl)-24-(1-oxopentoxy)-26,27-cyclo-9,10-secocholesta-5,7,10(19)-triene-1,3-diol

(5Z,7E)-(1S,3R,24R)-25-(5-butylthiazole-2-yl)-24-(1-oxopentoxy)-26,27-cyclo-9,10-secocholesta-5,7,10(19)-triene-1,3-diol

(5Z,7E)-(1S,3R,24S)-24-benzoyloxy-25-(5-butylthiazole-2-yl)-26,27-cyclo-9,10-secocholesta-5,7,10(19)-triene-1,3-diol

(5Z,7E)-(1S,3R,24R)-24-benzoyloxy-25-(5-butylthiazole-2-yl)-26,27-cyclo-9,10-secocholesta-5,7,10(19)-triene-1,3-diol

(5Z,7E)-(1S,3R,24S)-25-(5-butylthiazole-2-yl)-26,27-cyclo-24-(4-pyridylcarbonyl)-9,10-secocholesta-5,7,10(19)-triene-1,3-diol

(5Z,7E)-(1S,3R,24R)-25-(5-butylthiazole-2-yl)-26,27-cyclo-24-(4-pyridylcarbonyl)-9,10-secocholesta-5,7,10(19)-triene-1,3-diol

(5Z,7E)-(1S,3R,24S)-25-(5-butylthiazole-2-yl)-24-[[(ethylamino)carbonyl]oxy]-26,27-cyclo-9,10-secocholesta-5,7,10(19)-triene-1,3-diol

(5Z,7E)-(1S,3R,24R)-25-(5-butylthiazole-2-yl)-24-[[(ethylamino)carbonyl]oxy]-26,27-cyclo-9,10-secocholesta-5,7,10(19)-triene-1,3-diol

(5Z,7E)-(1S,3R,24S)-25-(5-butylthiazole-2-yl)-24-[[(methylamino)carbony]loxy]-26,27-cyclo-9,10-secocholesta-5,7,10(19)-triene-1,3-diol

(5Z,7E)-(1S,3R,24R)-25-(5-butylthiazole-2-yl)-24-[[(methylamino)carbonyl]oxy]-26,27-cyclo-9,10-secocholesta-5,7,10(19)-triene-1,3-diol

(5Z,7E)-(1S,3R,24S)-25-(5-butylthiazole-2-yl)-24-(3-hydroxy-2-methyl-1-oxopropoxy)-26,27-cyclo-9,10-secocholesta-5,7,10(19)-triene-1,3-diol

(5Z,7E)-(1S,3R,24R)-25-(5-butylthiazole-2-yl)-24-(3-hydroxy-2-methyl-1-oxopropoxy)-26,27-cyclo-9,10-secocholesta-5,7,10(19)-triene-1,3-diol

(5Z,7E)-(1S,3R,24S)-25-(5-butylthiazole-2-yl)-24-(2-methyl-1-oxopropoxy)-26,27-cyclo-9,10-secocholesta-5,7,10(19)-triene-1,3-diol

(5Z,7E)-(1S,3R,24R)-25-(5-butylthiazole-2-yl)-24-(2-methyl-1-oxopropoxy)-26,27-cyclo-9,10-secocholesta-5,7,10(19)-triene-1,3-diol

(5Z,7E)-(1S,3R,24S)-25-(5-butylthiazole-2-yl)-24-[[((pentafluorophenyl)amino)-carbonyl]oxy]-26,27-cyclo-9,10-secocholesta-5,7,10(19)-triene-1,3-diol

(5Z,7E)-(1S,3R,24R)-25-(5-butylthiazole-2-yl)-24-[[((pentafluorophenyl)amino)-carbonyl]oxy]-26,27-cyclo-9,10-secocholesta-5,7,10(19)-triene-1,3-diol

(5Z,7E)-(1S,3R,24S)-25-(5-butylthiazole-2-yl)-24-[[(2,2-dimethyl-3-hydroxy)-1-oxopropyl]oxy]-26,27-cyclo-9,10-secocholesta-5,7,10(19)-triene-1,3-diol

(5Z,7E)-(1S,3R,24R)-25-(5-butylthiazole-2-yl)-24-[[(2,2-dimethyl-3-hydroxy)-1-oxopropyl]oxy]-26,27-cyclo-9,10-secocholesta-5,7,10(19)-triene-1,3-diol

(5Z,7E,22E)-(1S,3R,24S)-25-(5-butylthiazole-2-yl)-26,27-cyclo-9,10-secocholesta-5,7,10(19),22-tetraene-1,3,24-triol tris-isonicotinate

(5Z,7E,22E)-(1S,3R,24R)-25-(5-butylthiazole-2-yl)-26,27-cyclo-9,10-secocholesta-5,7,10(19),22-tetraene-1,3,24-triol tris-isonicotinate

(5Z,7E,22E)-(1S,3R,24S)-25-(5-butylthiazole-2-yl)-26,27-cyclo-9,10-secocholesta-5,7,10(19),22-tetraene-1,3,24-triol tribenzoate

(5Z,7E,22E)-(1S,3R,24R)-25-(5-butylthiazole-2-yl)-26,27-cyclo-9,10-secocholesta-5,7,10(19),22-tetraene-1,3,24-triol tribenzoate

(5Z,7E)-(1S,3R,24S)-24-acetoxy-25-(4-methylthiazole-2-yl)-26,27-cyclo-9,10-secocholesta-5,7,10(19)-triene-1,3-diol

(5Z,7E)-(1S,3R,24R)-24-acetoxy-25-(-methylthiazole-2-yl)-26,27-cyclo-9,10-secocholesta-5,7,10(19)-triene-1,3-diol

(5Z,7E)-(1S,3R,24S)-24-(2,2-dimethyl-1-oxopropoxy)-25-(4-methylthiazole-2yl)-26,27-cyclo-9,10-secocholesta-5,7,10(19)-triene-1,3-diol

(5Z,7E)-(1S,3R,24R)-24-(2,2-dimethyl-1-oxopropoxy)-25-(4-methylthiazole-2-yl)-26,27-cyclo-9,10-secocholesta-5,7,10(19)-triene-1,3-diol

(5Z,7E)-(1S,3R,24S)-25-(4-methylthiazole-2-yl)-24-(1-oxobutoxy)-26,27-cyclo-9,10-secocholesta-5,7,10(19)-triene-1,3-diol

(5Z,7E)-(1S,3R,24R)-25-(4-methylthiazole-2-yl)-24-(1-oxobutoxy)-26,27-cyclo-9,10-secocholesta-5,7,10(19)-triene-1,3-diol

(5Z,7E)-(1S,3R,24S)-24-(2-methyl-1-oxobutoxy)-25-(4-methylthiazole-2-yl)-26,27-cyclo-9,10-secocholesta-5,7,10(19)-triene-1,3-diol

(5Z,7E)-(1S,3R,24R)-24-(2-methyl-1-oxobutoxy)-25-(4-methylthiazole-2-yl)-26,27-cyclo-9,10-secocholesta-5,7,10(19)-triene-1,3-diol

(5Z,7E)-(1S,3R,24S)-25-(4-methylthiazole-2-yl)-24-(1-oxopropoxy)-26,27-cyclo-9,10-secocholesta-5,7,10(19)-triene-1,3-diol

(5Z,7E)-(1S,3R,24R)-25-(4-methylthiazole-2-yl)-24-(1-oxopropoxy)-26,27-cyclo-9,10-secocholesta-5,7,10(19)-triene-1,3-diol

(5Z,7E)-(1S,3R,24S)-25-(4-methylthiazole-2-yl)-24-(1-oxopentyloxy)-26,27-cyclo-9,10-secocholesta-5,7,10(19)-triene-1,3-diol

(5Z,7E)-(1S,3R,24R)-25-(4-methylthiazole-2-yl)-24-(1-oxopentoxy)-26,27-cyclo-9,10-secocholesta-5,7,10(19)-triene-1,3-diol

(5Z,7E)-(1S,3R,24S)-24-benzoyloxy-25-(4-methylthiazole-2-yl)-26,27-cyclo-9,10-secocholesta-5,7,10(19)-triene-1,3-diol

(5Z,7E)-(1S,3R,24R)-24-benzoyloxy-25-(4-methylthiazole-2-yl)-26,27-cyclo-9,10-secocholesta-5,7,10(19)-triene-1,3-diol

(5Z,7E)-(1S,3R,24S)-25-(4-methylthiazole-2-yl)-26,27-cyclo-24-(4-pyridylcarbonyl) -9,10-secocholesta-5,7,10(19)-triene-1,3-diol

(5Z,7E)-(1S,3R,24R)-25-(4-methylthiazole-2-yl)-26,27-cyclo-24-(4-pyridylcarbonyl) -9,10-secocholesta-5,7,10(19)-triene-1,3-diol

(5Z,7E)-(1S,3R,24S)-24-[[(ethylamino)carbonyl]oxy]-25-(4-methylthiazole-2-yl)-26,27-cyclo-9,10-secocholesta-5,7,10(19)-triene-1,3-diol

(5Z,7E)-(1S,3R,24R)-24-[[(ethylamino)carbonyl]oxy]-25-(4-methylthiazole-2-yl)-26,27-cyclo-9,10-secocholesta-5,7,10(19)-triene-1,3-diol

(5Z,7E)-(1S,3R,24S)-24-[[(methylamino)carbonyl]oxy]-25-(4-methylthiazole-2-yl)-26,27-cyclo-9,10-secocholesta-5,7,10(19)-triene-1,3-diol

(5Z,7E)-(1S,3R,24R)-24-[[(methylamino)carbonyl]oxy]-25-(4-methylthiazole-2-yl)-26,27-cyclo-9,10-secocholesta-5,7,10(19)-triene-1,3-diol

(5Z,7E)-(1S,3R,24S)-24-(3-hydroxy-2-methyl-1-oxopropoxy)-25-(4-methyl-thiazole-2-yl)-26,27-cyclo-9,10-secocholesta-5,7,10(19)-triene-1,3-diol

(5Z,7E)-(1S,3R,24R)-24-(3-hydroxy-2-methyl-1-oxopropoxy)-25-(4-methyl-thiazole-2-yl)-26,27-cyclo-9,10-secocholesta-5,7,10(19)-triene-1,3-diol

(5Z,7E)-(1S,3R,24S)-24-(2-methyl-1-oxopropoxy)-25-(4-methylthiazole-2-yl)-26,27-cyclo-9,10-secocholesta-5,7,10(19)-triene-1,3-diol

(5Z,7E)-(1S,3R,24R)-24-(2-methyl-1-oxopropoxy)-25-(4-methylthiazole-2-yl)-26,27-cyclo-9,10-secocholesta-5,7,10(19)-triene-1,3-diol

(5Z,7E)-(1S,3R,24S)-25-(4-methylthiazole-2-yl)-(24-[[((pentafluorophenyl)amino)-carbonyl]oxy]-26,27-cyclo-9,10-secocholesta-5,7,10(19)-triene-1,3-diol

(5Z,7E)-(1S,3R,24R)-25-(4-methylthiazole-2-yl)-24-[[((pentafluorophenyl)amino)-carbonyl]oxy]-26,27-cyclo-9,10-secocholesta-5,7,10(19)-triene-1,3-diol

(5Z,7E)-(1S,3R,24S)-24-[[(2,2-dimethyl-3-hydroxy)-1-oxopropyl]oxy]-25-(4-methylthiazole-2-yl)-26,27-cyclo-9,10-secocholesta-5,7,10(19)-triene-1,3-diol

(5Z,7E)-(1S,3R,24R)-24-[[(2,2-dimethyl-3-hydroxy)-1-oxopropyl]oxy]-25-(4-methylthiazole-2-yl)-26,27-cyclo-9,10-secocholesta-5,7,10(19)-triene-1,3-diol

(5Z,7E,22E)-(1S,3R,24S)-25-(4-methylthiazole-2-yl)-26,27-cyclo-9,10-secocholesta-5,7,10(19),22-tetraene-1,3,24-triol tris-isonicotinate

(5Z,7E,22E)-(1S,3R,24R)-25-(4-methylthiazole-2-yl)-26,27-cyclo-9,10-secocholesta-5,7,10(19),22-tetraene-1,3,24-triol tris-isonicotinate

(5Z,7E,22E)-(1S,3R,24S)-25-(4-methylthiazole-2-yl)-26,27-cyclo-9,10-secocholesta-5,7,10(19),22-tetraene-1,3,24-triol tribenzoate

(5Z,7E,22E)-(1S,3R,24R)-25-(4-methylthiazole-2-yl)-26,27-cyclo-9,10-secocholesta-5,7,10(19),22-tetraene-1,3,24-triol tribenzoate

(5Z,7E)-(1S,3R,24S)-24-acetoxy-25-(4-methyloxazole-2-yl)-26,27-cyclo-9,10-secocholesta-5,7,10(19)-triene-1,3-diol

(5Z,7E)-(1S,3R,24R)-24-acetoxy-25-(4-methyloxazole-2-yl)-26,27-cyclo-9,10-secocholesta-5,7,10(19)-triene-1,3-diol

(5Z,7E)-(1S,3R,24S)-24-(2,2-dimethyl-1-oxopropoxy)-25-(4-methyloxazole-2-yl)-26,27-cyclo-9,10-secocholesta-5,7,10(19)-triene-1,3-diol

(5Z,7E)-(1S,3R,24R)-24-(2,2-dimethyl-1-oxopropoxy)-25-(4-methyloxazole-2-yl)-26,27-cyclo-9,10-secocholesta-5,7,10(19)-triene-1,3-diol

(5Z,7E)-(1S,3R,24S)-25-(4-methyloxazole-2-yl)-24-(1-oxobutoxy)-26,27-cyclo-9,10-secocholesta-5,7,10(19)-triene-1,3-diol

(5Z,7E)-(1S,3R,24R)-25-(4-methyloxazole-2-yl)-24-(1-oxobutoxy)-26,27-cyclo-9,10-secocholesta-5,7,10(19)-triene-1,3-diol

(5Z,7E)-(1S,3R,24S)-25-(4-methyloxazole-2-yl)-24-(2-methyl-1-oxobutoxy)-26,27-cyclo-9,10-secocholesta-5,7,10(19)-triene-1,3-diol

(5Z,7E)-(1S,3R,24R)-25-(4-methyloxazole-2-yl)-24-(2-methyl-1-oxobutoxy)-26,27-cyclo-9,10-secocholesta-5,7,10(19)-triene-1,3-diol

(5Z,7E)-(1S,3R,24S)-25-(4-methyloxazole-2-yl)-24-(1-oxopropoxy)-26,27-cyclo-9,10-secocholesta-5,7,10(19)-triene-1,3-diol

(5Z,7E)-(1S,3R,24R)-25-(4-methyloxazole-2-yl)-24-(1-oxopropoxy)-26,27-cyclo-9,10-secocholesta-5,7,10(19)-triene-1,3-diol

(5Z,7E)-(1S,3R,24S)-25-(4-methyloxazole-2-yl)-24-(1-oxopentoxy)-26,27-cyclo-9,10-secocholesta-5,7,10(19)-triene-1,3-diol

(5Z,7E)-(1S,3R,24R)-25-(4-methyloxazole-2-yl)-24-(1-oxopentoxy)-26,27-cyclo-9,10-secocholesta-5,7,10(19)-triene-1,3-diol

(5Z,7E)-(1S,3R,24S)-24-benzoyloxy-25-(4-methyloxazole-2-yl)-26,27-cyclo-9,10-secocholesta-5,7,10(19)-triene-1,3-diol

(5Z,7E)-(1S,3R,24R)-24-benzoyloxy-25-(4-methyloxazole-2-yl)-26,27-cyclo-9,10-secocholesta-5,7,10(19)-triene-1,3-diol

(5Z,7E)-(1S,3R,24S)-25-(4-methyloxazole-2-yl)-26,27-cyclo-24-(4-pyridylcarbonyl)-9,10-secocholesta-5,7,10(19)-triene-1,3-diol

(5Z,7E)-(1S,3R,24R)-25-(4-methyloxazole-2-yl)-26,27-cyclo-24-(4-pyridylcarbonyl)-9,10-secocholesta-5,7,10(19)-triene-1,3-diol

(5Z,7E)-(1S,3R,24S)-24-[[(ethylamino)carbonyl]oxy]-25-(4-methyloxazole-2-yl)-26,27-cyclo-9,10-secocholesta-5,7,10(19)-triene-1,3-diol

(5Z,7E)-(1S,3R,24R)-24-[[(ethylamino)carbonyl]oxy]-25-(4-methyloxazole-2-yl)-26,27-cyclo-9,10-secocholesta-5,7,10(19)-triene-1,3-diol

(5Z,7E)-(1S,3R,24S)-24-[[(methylamino)carbonyl]oxy]-25-(4-methyloxazole-2-yl)-26,27-cyclo-9,10-secocholesta-5,7,10(19)-triene-1,3-diol

(5Z,7E)-(1S,3R,24R)-24-[[(methylamino)carbonyl]oxy]-25-(4-methyloxazole-2-yl)-26,27-cyclo-9,10-secocholesta-5,7,10(19)-triene-1,3-diol

(5Z,7E)-(1S,3R,24S)-24-(3-hydroxy-2-methyl-1-oxopropoxy)-25-(4-methyl-oxazole-2-yl)-26,27-cyclo-9,10-secocholesta-5,7,10(19)-triene-1,3-diol

(5Z,7E)-(1S,3R,24R)-24-(3-hydroxy-2-methyl-1-oxopropoxy)-25-(4-methyl-oxazole-2-yl)-26,27-cyclo-9,10-secocholesta-5,7,10(19)-triene-1,3-diol

(5Z,7E)-(1S,3R,24S)-25-(4-methyloxazole-2-yl)-24-(2-methyl-1-oxopropoxy)-26,27-cyclo-9,10-secocholesta-5,7,10(19)-triene-1,3-diol

(5Z,7E)-(1S,3R,24R)-25-(4-methyloxazole-2-yl)-24-(2-methyl-1-oxopropoxy)-26,27-cyclo-9,10-secocholesta-5,7,10(19)-triene-1,3-diol

(5Z,7E)-(1S,3R,24S)-25-(4-methyloxazole-2-yl)-(24-[[((pentafluorophenyl)amino)carbonyl]oxy]-26,27-cyclo-9,10-secocholesta-5,7,10(19)-triene-1,3-diol

(5Z,7E)-(1S,3R,24R)-25-(4-methyloxazole-2-yl)-24-[[((pentafluorophenyl)amino)-carbonyl]oxy]-26,27-cyclo-9,10-secocholesta-5,10(19)-triene-1,3-diol

(5Z,7E)-(1S,3R,24S)-24-[[(2,2-dimethyl-3-hydroxy)-1-oxopropyl]oxy]-25-(4-methyloxazole-2-yl)-26,27-cyclo-9,10-secocholesta-5,7,10(19)-triene-1,3-diol

(5Z,7E)-(1S,3R,24R)-24-[[(2,2-dimethyl-3-hydroxy)-1-oxopropyl]oxy]-25-(4-methyloxazole-2-yl)-26,27-cyclo-9,10-secocholesta-5,7,10(19)-triene-1,3-diol

(5Z,7E,22E)-(1S,3R,24S)-25-(4-methyloxazole-2-yl)-26,27-cyclo-9,10-secocholesta-5,7,10(19),22-tetraene-1,3,24-triol tris-isonicotinate

(5Z,7E,22E)-(1S,3R,24R)-25-(4-methyloxazole-2-yl)-26,27-cyclo-9,10-secocholesta-5,7,10(19),22-tetraene-1,3,24-triol tris-isonicotinate

(5Z,7E,22E)-(1S,3R,24S)-25-(4-methyloxazole-2-yl)-26,27-cyclo-9,10-secocholesta-5,7,10(19),22-tetraene-1,3,24-triol tribenzoate

(5Z,7E,22E)-(1S,3R,24R)-25-(4-methyloxazole-2-yl)-26,27-cyclo-9,10-secocholesta-5,7,10(19),22-tetraene-1,3,24-triol tribenzoate

(5Z,7E)-(1S,3R,24S)-24-acetoxy-26,27-cyclo-25-(3-propyl-1,2,4-oxadiazole-5-yl)-9,10-secocholesta-5,7,10(19)-triene-1,3-diol

(5Z,7E)-(1S,3R,24R)-24-acetoxy-26,27-cyclo-25-(3-propyl-1,2,4-oxadiazole-5-yl)-9,10-secocholesta-5,7,10(19)-triene-1,3-diol

(5Z,7E)-(1S,3R,24S)-24-(2,2-dimethyl-1-oxopropoxy)-25-(3-propyl-1,2,4-oxadiazole-5-yl)-26,27-cyclo-9,10-secocholesta-5,7,10(19)-triene-1,3-diol

(5Z,7E)-(1S,3R,24R)-24-(2,2-dimethyl-1-oxopropoxy)-25-(3-propyl-1,2,4-oxadiazole-5-yl)-26,27-cyclo-9,10-secocholesta-5,7,10(19)-triene-1,3-diol

(5Z,7E)-(1S,3R,24S)-24-(1-oxobutoxy)-25-(3-propyl-1,2,4-oxadiazole-5-yl)-26,27-cyclo-9,10-secocholesta-5,7,10(19)-triene-1,3-diol

(5Z,7E)-(1S,3R,24R)-24-(1-oxobutoxy)-25-(3-propyl-1,2,4-oxadiazole-5-yl)-26,27-cyclo-9,10-secocholesta-5,7,10(19)-triene-1,3-diol

(5Z,7E)-(1S,3R,24S)-24-(2-methyl-1-oxobutoxy)-25-(3-propyl-1,2,4-oxadiazole-5-yl)-26,27-cyclo-9,10-secocholesta-5,7,10(19)-triene-1,3-diol

(5Z,7E)-(1S,3R,24R)-24-(2-methyl-1-oxobutoxy)-25-(3-propyl-1,2,4-oxadiazole-5-yl)-26,27-cyclo-9,10-secocholesta-5,7,10(19)-triene-1,3-diol

(5Z,7E)-(1S,3R,24S)-24-(1-oxopropoxy)-25-(3-propyl-1,2,4-oxadiazole-5-yl)-26,27-cyclo-9,10-secocholesta-5,7,10(19)-triene-1,3-diol

(5Z,7E)-(1S,3R,24R)-24-(1-oxopropoxy)-25-(3-propyl-1,2,4-oxadiazole-5-yl)-26,27-cyclo-9,10-secocholesta-5,7,10(19)-triene-1,3-diol

(5Z,7E)-(1S,3R,24S)-24-(1-oxopentoxy)-25-(3-propyl-1,2,4-oxadiazole-5-yl)-26,27-cyclo-9,10-secocholesta-5,7,10(19)-triene-1,3-diol

(5Z,7E)-(1S,3R,24R)-24-(1-oxopentoxy)-25-(3-propyl-1,2,4-oxadiazole-5-yl)-26,27-cyclo-9,10-secocholesta-5,7,10(19)-triene-1,3-diol

(5Z,7E)-(1S,3R,24S)-24-benzoyloxy-25-(3-propyl-1,2,4-oxadiazole-5-yl)-26,27-cyclo-9,10-secocholesta-5,7,10(19)-triene-1,3-diol

(5Z,7E)-(1S,3R,24R)-24-benzoyloxy-25-(3-propyl-1,2,4-oxadiazole-5-yl)-26,27-cyclo-9,10-secocholesta-5,7,10(19)-triene-1,3-diol

(5Z,7E)-(1S,3R,24S)-25-(3-propyl-1,2,4-oxadiazole-5-yl)-24-(4-pyridylcarbonyl)-26,27-cyclo-9,10-secocholesta-5,7,10(19)-triene-1,3-diol

(5Z,7E)-(1S,3R,24R)-25-(3-propyl-1,2,4-oxadiazole-5-yl)-24-(4-pyridylcarbonyl)-26,27-cyclo-9,10-secocholesta-5,7,10(19)-triene-1,3-diol

(5Z,7E)-(1S,3R,24S)-24-[[(ethylamino)carbonyl]oxy]-25-(3-propyl-1,2,4-oxadiazole-5-yl)-26,27-cyclo-9,10-secocholesta-5,7,10(19)-triene-1,3-diol

(5Z,7E)-(1S,3R,24R)-24-[[(ethylamino)carbonyl]oxy]-25-(3-propyl-1,2,4-oxadiazole-5-yl)-26,27-cyclo-9,10-secocholesta-5,7,10(19)-triene-1,3-diol

(5Z,7E)-(1S,3R,24S)-24-[[(methylamino)carbonyl]oxy]-25-(3-propyl-1,2,4-oxadiazole-5-yl)-26,27-cyclo-9,10-secocholesta-5,7,10(19)-triene-1,3-diol

(5Z,7E)-(1S,3R,24R)-24-[[(methylamino)carbonyl]oxy]-25-(3-propyl-1,2,4-oxadiazole-5-yl)-26,27-cyclo-9,10-secocholesta-5,7,10(19)-triene-1,3-diol

(5Z,7E)-(1S,3R,24S)-24-(3-hydroxy-2-methyl-1-oxopropoxy)-25-(3-propyl-1,2,4-oxadiazole-5-yl)-26,27-cyclo-9,10-secocholesta-5,7,10(19)-triene-1,3-diol

(5Z,7E)-(1S,3R,24R)-24-(3-hydroxy-2-methyl-1-oxopropoxy)-25-(3-propyl-1,2,4-oxadiazole-5-yl)-26,27-cyclo-9,10-secocholesta-5,7,10(19)-triene-1,3-diol

(5Z,7E)-(1S,3R,24S)-24-(2-methyl-1-oxopropoxy)-25-(3-propyl-1,2,4-oxadiazole-5-yl-26,27-cyclo-9,10-secocholesta-5,7,10(19)-triene-1,3-diol

(5Z,7E)-(1S,3R,24R)-24-(2-methyl-1-oxopropoxy)-25-(3-propyl-1,2,4-oxadiazole-5-yl-26,27-cyclo-9,10-secocholesta-5,7,10(19)-triene-1,3-diol

(5Z,7E)-(1S,3R,24S)-24-[[((pentafluorophenyl)amino)-carbonyl]oxy]-25-(3-propyl-1,2,4-oxadiazole-5-yl)-26,27-cyclo-9,10-secocholesta-5,7,10(19)-triene-1,3-diol

(5Z,7E)-(1S,3R,24R)-)-24-[[((pentafluorophenyl)amino)-carbonyl]oxy]-25-(3-propyl-1,2,4-oxadiazole-5-yl)-26,27-cyclo-9,10-secocholesta-5,7,10(19)-triene-1,3-diol

(5Z,7E)-(1S,3R,24S)-24-[[(2,2-dimethyl-3-hydroxy)-1-oxopropyl]oxy]-25-(3-propyl-1,2,4-oxadiazole-5-yl)-26,27-cyclo-9,10-secocholesta-5,7,10(19)-triene-1,3-diol

(5Z,7E)-(1S,3R,24R)-24-[[(2,2-dimethyl-3-hydroxy)-1-oxopropyl]oxy]-25-(3-propyl-1,2,4-oxadiazole-5-yl)-26,27-cyclo-9,10-secocholesta-5,7,10(19)-triene-1,3-diol

(5Z,7E,22E)-(1S,3R,24S)-25-(3-propyl-1,2,4-oxadiazole-5-yl)-26,27-cyclo-9,10-secocholesta-5,7,10(19),22-tetraene-1,3,24-triol tris-isonicotinate

(5Z,7E,22E)-(1S,3R,24R)-25-(3-propyl-1,2,4-oxadiazole-5-yl)-26,27-cyclo-9,10-secocholesta-5,7,10(19),22-tetraene-1,3,24-triol tris-isonicotinate

(5Z,7E,22E)-(1S,3R,24S)-25-(3-propyl-1,2,4-oxadiazole-5-yl)-26,27-cyclo-9,10-secocholesta-5,7,10(19),22-tetraene-1,3,24-triol tribenzoate

(5Z,7E,22E)-(1S,3R,24R)-25-(3-propyl-1,2,4-oxadiazole-5-yl)-26,27-cyclo-9,10-secocholesta-5,7,10(19),22-tetraene-1,3,24-triol tribenzoate

(5Z,7E)-(1S,3R,24S)-24-acetoxy-25-(4-propylimidazole-2-yl)-26,27-cyclo-9,10-secocholesta-5,7,10(19)-triene-1,3-diol

(5Z,7E)-(1S,3R,24R)-24-acetoxy-25-(4-propylimidazole-2-yl)-26,27-cyclo-9,10-secocholesta-5,7,10(19)-triene-1,3-diol

(5Z,7E)-(1S,3R,24S)-24-(2,2-dimethyl-1-oxopropoxy)-25-(4-propylimidazole-2-yl)-26,27-cyclo-9,10-secocholesta-5,7,10(19)-triene-1,3-diol

(5Z,7E)-(1S,3R,24R)-24-(2,2-dimethyl-1-oxopropoxy)-25-(4-propylamidazole-2-yl)-26,27-cyclo-9,10-secocholesta-5,7,10(19)-triene1,3-diol

(5Z,7E)-(1S,3R,24S)-24-(1-oxobutoxy)-25-(4-propylimidazole-2-yl)-26,27-cyclo-9,10secocholesta-5,7,10(19)-triene-1,3-diol

(5Z,7E)-(1S,3R,24R)-24-(1-oxobutoxy)-25-(4-propylimidazole-2-yl)-26,27-cyclo-9,10secocholesta-5,7,10(19)-triene-1,3-diol

(5Z,7E)-(1S,3R,24S)-24-(2-methyl-1-oxobutoxy)-25-(4-propylimidazole-2-yl)-26,27-cyclo-9,10-secocholesta-5,7,10(19)-triene-1,3-diol

(5Z,7E)-(1S,3R,24R)-24-(2-methyl-1-oxobutoxy)-25-(4-propylimidazole-2-yl)-26,27-cyclo-9,10-secocholesta-5,7,10(19)-triene-1,3-diol

(5Z,7E)-(1S,3R,24S)-24-(1-oxopropoxy)-25-(4-propylimidazole-2-yl)-26,27-cyclo-9,10-secocholesta-5,7,10(19)-triene-1,3-diol

(5Z,7E)-(1S,3R,24R)-24-(1-oxopropoxy)-25-(4-propylamidazole-2-yl)-26,27-cyclo-9,10-secocholesta-5,7,10(19)-triene-1,3-diol

(5Z,7E)-(1S,3R,24S)-24-(1-oxopentoxy)-25-(4-propylimidazole-2-yl)-26,27-cyclo-9,10-secocholesta-5,7,10(19)-triene-1,3-diol

(5Z,7E)-(1S,3R,24R)-24-(1-oxopentoxy)-25-(4-propylimidazole-2-yl)-26,27-cyclo-9,10-secocholesta-5,7,10(19)-triene-1,3-diol

(5Z,7E)-(1S,3R,24S)-24-benzoyloxy-25-(4-propylimidazole-2-yl)-26,27-cyclo-9,10-secocholesta-5,7,10(19)-triene-1,3-diol

(5Z,7E)-(1S,3R,24R)-24-benzoyloxy-25-(4-propylimidazole-2-yl)-26,27-cyclo-9,10-secocholesta-5,7,10(19)-triene-1,3-diol

(5Z,7E)-(1S,3R,24S)-25-(4-propylimidazole-2-yl)-26,27-cyclo-24-(4-pyridylcarbonyl)-9,10-secocholesta-5,7,10(19)-triene-1,3-diol

(5Z,7E)-(1S,3R,24R)-25-(4-propylimidazole-2-yl)-26,27-cyclo-24-(4-pyridylcarbonyl-9,10-secocholesta-5,7,10(19)-triene-1,3-diol

(5Z,7E)-(1S,3R,24S)-24-[[(ethylamino)carbonyl]oxy]-25-(4-propylimidazole-2-yl)-26,27-cyclo-24-9,10-secocholesta-5,7,10(19)-triene-1,3-diol

(5Z,7E)-(1S,3R,24R)-24-[[(ethylamino)carbonyl]oxy]-25-(4-propylimidazole-2-yl)-26,27-cyclo-9,10-secocholesta-5,7,10(19)-triene-1,3-diol

(5Z,7E)-(1S,3R,24S)-24-[[(methylamino)carbonyl]oxy]-25-(4-propylimidazole-2-yl)-26,27-cyclo-9,10-secocholesta-5,7,10(19)-triene-1,3-diol

(5Z,7E)-(1S,3R,24R)-24-[[(methylamino)carbonyl]oxy]-25-(4-propylimidazole-2-yl)-26,27-cyclo-9,10-secocholesta-5,7,10(19)-triene-1,3-diol

(5Z,7E)-(1S,3R,24S)-24-(3-hydroxy-2-methyl-1-oxopropoxy)-25-(4-propylimidazole-2yl)-26,27-cyclo-9,10-secocholesta-5,7,10(19)-triene-1,3-diol

(5Z,7E)-(1S,3R,24R)-24-(3-hydroxy-2-methyl-1-oxopropoxy)-25-(4-propylimidazole-2yl)-26,27-cyclo-9,10-secocholesta-5,7,10(19)-triene-1,3-diol

(5Z,7E)-(1S,3R,24S)-24-(2-methyl-1-oxopropoxy)-25-(4-propylimidazole-2-yl)-26,27-cyclo-9,10-secocholesta-5,7,10(19)-triene-1,3-diol

(5Z,7E)-(1S,3R,24R)-24-(2-methyl-1-oxopropoxy)-25-(4-propylamidazole-2-yl)-26,27-cyclo-9,10-secocholesta-5,7,10(19)-triene-1,3-diol

(5Z,7E)-(1S,3R,24S)-24-[[((pentafluorophenyl)amino)-carbonyl]oxy]-25-(4-propylimidazole-2-yl)-26,27-cyclo-9,10-secocholesta-5,7,10(19)-triene-1,3-diol

(5Z,7E)-(1S,3R,24R)-24-[[((pentafluorophenyl)amino)-carbonyl]oxy]-25-(4-propylimidazole-2-yl)-26,27-cyclo-9,10-secocholesta-5,7,10(19)-triene-1,3-diol

(5Z,7E)-(1S,3R,24S)-24-[[(2,2-dimethyl-3-hydroxy)-1-oxopropyl]oxy]-25-(4-propylimidazole-2-yl)-26,27-cyclo-9,10-secocholesta-5,7,10(19)-triene-1,3-diol

(5Z,7E)-(1S,3R,24R)-24-[[(2,2-dimethyl-3-hydroxy)-1-oxopropyl]oxy]-25-(4-propylimidazole-2-yl)-26,27-cyclo-9,10-secocholesta-5,7,10(19)-triene-1,3-diol

(5Z,7E,22E)-(1S,3R,24S)-25-(4-propylimidazole-2-yl)-26,27-cyclo-9,10-secocholesta-5,7,10(19),22-tetraene-1,3,24-triol tris-isonicotinate

(5Z,7E,22E)-(1S,3R,24R)-25-(4-propylimidazole-2-yl)-26,27-cyclo-9,10-secocholesta-5,7,10(19),22-tetraene-1,3,24-triol tris-isonicotinate

(5Z,7E,22E)-(1S,3R,24S)-25-(4-propylimidazole-2-yl)-26,27-cyclo-9,10-secocholesta-5,7,10(19),22-tetraene-1,3,24-triol tris-isonicotinate

(5Z,7E,22E)-(1S,3R,24S)-25-(4-propylimidazole-2-yl)-26,27-cyclo-9,10-secocholesta-5,7,10(19),22-tetraene-1,3,24-triol tribenzoate

(5Z,7E,22E)-(1S,3R,24R)-25-(4-propylimidazole-2-yl)-26,27-cyclo-9,10-secocholesta-5,7,10(19),22-tetraene-1,3,24-triol tribenzoate

The substances according to the invention have a considerably higher metabolic stability especially in liver microsomes than the structurally related compounds of the prior art and are therefore suitable in a special way for systemic administrations.

In addition, they are largely inactive in vitro, while they have a surprisingly high activity in vivo.

Relative to the structurally related compounds of the prior art, the substances according to the invention are also characterized in that they do not show any action on cell differentiation, while in vivo prominent immunoregulative effects are observed. The induction of undesirable hypercalcemia is carried out only at considerably higher dosages than in the compounds of the prior art.

Determination of Biological Activity

The vitamin D activity of the substances according to the invention is determined with the aid of the calcitriol-receptor test. It is carried out using a protein extract from the intestines of juvenile pigs. Receptor-containing protein extract is incubated in a test tube with ³H-calcitriol (5×10⁻¹⁰ mol/l) in a reaction volume of 0.27 ml in the absence and in the presence of test substances for two hours at 4° C. To separate free and receptor-bonded calcitriol, a charcoal-dextran absorption is carried out. To this end, 250 il of a charcoal-dextran suspension is fed to each test tube and incubated at 4° C. for 20 minutes. Then, the samples are centrifuged at 10,000 g for 5 minutes at 4° C. The supernatant is decanted and measured in a á-counter after 1 hour of equilibration in Picofluor 15™.

The competition curves that are obtained at various concentrations of test substance as well as of reference substance (unlabeled calcitriol) at constant concentration of the reference substance (³H-calcitriol) are placed in relation to one another, and a competition factor (KF) is determined.

It is defined as a quotient of the concentrations of the respective test substance and the reference substance, which are necessary for 50% competition:

KF=Concentration of test substance at 50% competition

Concentration of reference substance at 50% competition

To determine the acute hypercalcemic action of various calcitriol derivatives, the test that is described below is carried out:

The action of control (solution base), reference substance (1,25-dihydroxy vitamin D₃=calcitriol) and test substance is tested in each case after one-time subcutaneous administration in groups of 10 healthy male rats (140-170 g). During the testing time, the rats are kept in special cages to determine the excretion of water and mineral substances. Urine is collected in 2 fractions (0-16 hours and 16-22 hours). An oral dose of calcium (0.1 mmol of calcium in 6.5% alpha-hydroxypropyl cellulose, 5 ml/animal) replaces at 1600 hours the calcium intake that is lacking by food deprivation. At the end of the test, the animals are sacrificed by decapitation and exsanguinated to determine the serum-calcium values. For the primary screening test in vivo, an individual standard dose (200 ig/kg) is tested. For selected substances, the result is supported by establishing a dose-effect relation.

A hypercalcemic action is shown in serum-calcium level values that are higher than in the control.

The significance of differences occurring between substance groups and controls as well as between test substance and reference substance are supported with suitable statistical processes. The result is indicated as dose ratio DR (DR=factor of test substance dose/reference substance dose for comparable actions).

The differentiation-stimulating action of calcitriol analogs is also detected quantitatively.

It is known in the literature [D. J. Mangelsdorf et al., J. Cell. Biol. 98, 391 (1984)] that the treatment of human leukemia cells (promyelocyte cell line HL 60) in vitro with calcitriol induces the differentiation of cells to macrophages.

HL 60 cells are cultivated in tissue culture medium (RPMI 10% fetal calf serum) at 37° C. in an atmosphere of 5% CO₂ in air.

For substance testing, the cells are centrifuged off, and 2.0×10⁵ cells/ml in phenol red-free tissue culture medium are taken up. The test substances are dissolved in ethanol and diluted to the desired concentration with tissue culture medium without phenol red. The dilution stages are mixed with the cell suspension at a ratio of 1:10, and 100 il each of this cell suspension that is mixed with substance is pipetted into an indentation of a 96-hole plate. For control, a cell suspension is mixed analogously with the solvent.

After incubation for 96 hours at 37° C. in 5% CO₂ in air, 100 il of an NBT-TPA solution (nitro blue tetrazolium (NBT), final concentration in the batch of 1 mg/ml, tetradecanoyl phorbolmyristate-13-acetate (TPA), final concentration in the batch of 2×10⁻⁷ mol/l) is pipetted into each indentation of the 96-hole plate in the cell suspension.

By incubation for 2 hours at 37° C. and 5% CO₂ in air, NBT is reduced to insoluble formazan because of the intracellular oxygen radical release, stimulated by TPA, in the cells that are differentiated to macrophages.

To complete the reaction, the indentations of the 96-hole plate are suctioned off, and the cells are affixed to the bottom of the plate by adding methanol and dried after affixing. To dissolve the intracellular formazan crystals that are formed, 100 il of potassium hydroxide (2 mol/l) and 100 il of dimethyl sulfoxide are pipetted into each indentation and ultrasonically treated for 1 minute. The concentration of formazan is measured by spectrophotometry at 650 nm.

As a yardstick for the differentiation induction of HL 60 cells to macrophages, the concentration of formed formazan applies. The result is indicated as a dose ratio (DR=factor of test substance dose/reference substance dose for comparable semi-maximum actions).

The results of the calcitriol-receptor test as well as the determination of the dose ratio of the differentiation induction of HL 60 cells and the dose ratio for hypercalcemia are summarized below:

Examples of test substances:

(5Z,7E)-(1S,3R,24S)-24-Acetoxy-25-(5-butyloxazole-2-yl)-26,27-cyclo-9,10-secocholesta-5,7,10(19)-triene-1,3-diol 3a

(5Z,7E)-(1S,3R,24R)-24-(Acetoxy)-25-(5-butyloxazole-2-yl)-26,27-cyclo-9,10-secocholesta-5,7,10(19)-triene-1,3-diol 3b

(5Z,7E)-(1S,3R,24S)-25-(5-Butyloxazole-2-yl)-24-(2,2-dimethyl-1-oxopropyl)-26,27-cyclo-9,10-secocholesta-5,7,10(19)-triene-1,3-diol 5a

(5Z,7E)-(1S,3R,24R)-25-(5-Butyloxazole-2-yl)-24-(2,2-dimethyl-1-oxopropyl)-26,27-cyclo-9,10-secocholesta-5,7,10(19)-triene-1,3-diol 5b

The compounds presented do not show any affinity to the vitamin D receptor and do not have any cell-differentiating activity in vitro.

The induction of a hypercalcemia is readily carried out only at very much higher doses than with calcitriol.

The following compounds from WO 97/41096 are contained in the table presented below for comparison:

(5Z,7E,22E)-(1S,3R,24R)-25-(5-Propyloxazole-2-yl)-26,27-cyclo-9,10-secocholesta-5,7,10(19),22-tetraene-1,3,24-triol (A)

(5Z,7E,22E)-(1S,3R,24R)-25-(5-Methyloxazole-2-yl)-26,27-cyclo-9,10-secocholesta-5,7,10(19),22-tetraene-1,3,24-triol (B)

(5Z,7E,22E)-(1S,3R,24R)-25-(5-Ethyloxazole-2-yl)-26,27-cyclo-9,10-secocholesta-5,7,10(19),22-tetraene-1,3,24-triol (C)

(5Z,7E,22E)-(1S,3R,24R)-25-(5-Pentyloxazole-2-yl)-26,27-cyclo-9,10-secocholesta-5,7,10(19),22-tetraene-1,3,24-triol (D)

| | KF | DR (HL 60) | DR (Ca) |
|---|---|---|---|
| 3a | >100 | >170 | >1000 |
| 3b | >100 | >170 | >1000 |
| 5a | >100 | >170 | >1000 |
| 5b | >100 | >170 | >1000 |
| A | 2 | 1.9 | 300 |
| B | 2 | 3.4 | >300 |
| C | 3 | 1.3 | >300 |
| D | 4 | >100 | >300 |
| Calcitriol | 1 | 1 | 1 |

The values in the table confirm the inactivity of compounds 3a, 3b, 5a, 5b in vitro in contrast to the comparison compounds of WO 97/41096.

In the animal model (DNFB-DTH), these substances, however, showed activity with respect to prevention of the formation of edemas without side effects.

In animal models that reflect immunosuppressive action (contact allergy in the mouse), however, the substances are highly effective with respect to the prevention of the formation of edemas without having side effects [K. M. Tramposch Agents and Actions Suppl. 58, 179-204 (1999)]. The model of the dinitrofluorobenzene (DNFB)-induced allergic contact allergy in the mouse represents an inflammatory skin disease with the background of an inflammatory reaction of the delayed type (delayed type hypersensitivity; DTH). The inflammatory phase of the contact allergy is triggered by a multi-phase reaction. A repeated contact with the topically effective contact allergen DNFB (25 il of a 0.5% (w/v) DNFB solution in acetone/olive oil 4:1 [v/v] on the shaved abdomen with a surface area of about 10 cm² on test days 0. and 1.) triggers an immune reaction, which results in the sensitization of the animals against the specific agent, without “visible” symptoms occurring. Only after a sensitization time of 4-5 days, in which populations of DNFB-specific lymphocytes of “memory” type have formed in the draining lymph nodes, can a renewed contact (20 il of a 0.5% (w/v) DNFB solution in acetone/olive oil 4:1 [v/v]) trigger an inflammatory skin reaction with the contact allergen at any skin area (here: on both ears, treated surface area/ear about 2 cm²). This second phase, which is also referred to as the trigger or “challenge,” ends in an eczematous lesion on the second contact site. The evaluation of the extent of the inflammatory reaction or the success of treatment is performed routinely 24 hours after the triggering, since at this time, the parameters that are to be analyzed (ear thickness and cell infiltration as a yardstick for the edema) have reached a plateau. The model of the DNFB-induced contact allergy allows the examination of the action of systemically or topically administered test substances with immunosuppressive/immunomodulating or inflammation-inhibiting properties. In the study on immunomodulating properties of calcitriol derivatives, the latter are administered once daily intragastrically in physiological common salt solution (0.9% NaCl) with an admixture of 0.085% Myrj 53) around the time of sensitization (beginning two days before and one day before sensitization, on the day of sensitization and on day 1 and 2 after sensitization). As side-effect parameters that are typical of vitamin D, the determination of the calcium level in the urine of the treated animals is used at the latest 24 hours after the last treatment with the substance. The calcium content is determined by flame-photometry. The significance of differences occurring between substance groups and controls as well as between test substance and reference substance are supported with suitable statistic processes. The therapeutic range of the test substances and calcitriol as a reference substance is indicated as a dose ratio DR (DR=factor of hypercalcemic threshold dose/immunosuppressive threshold dose).

| | DR (Hypercalcemic |
|---|---|
| | threshold dose/ |
| | immunosuppressive |
| | threshold dose) |
| 5b | 20,000 |
| (5Z,7E,22E)-(1S,3R,24R)-25-(5-Butyl- | 1,000 |
| oxazole-2-yI)-26,27-cyclo-9,10-secocholesta | |
| 5,7,10(19),22-tetraene-1,3,24-triol | |
| Calcitriol | 1 |

In summary, it is shown that the substances that are presented here (3a, 3b, 5a, 5b) are therapeutically active in vivo despite their deficient activity in in vitro test systems that are relevant for vitamin D derivatives. The conversion into biologically active substance therefore is carried out exclusively in vivo. Moreover, it is shown that the therapeutic range (immunosuppressive threshold dose versus hypercalcemic threshold dose) of these substances (Example 5b depicted by way of example for the substances presented) is clearly improved relative to compounds that have free hydroxy groups in the side chain.

By the reduced property of triggering a hypercalcemia, the substances according to the invention are suitable in a special way for the production of pharmaceutical agents for the treatment of diseases that are characterized by hyperproliferation and deficient cell differentiation. Included in these are, for example, hyperproliferative diseases of the skin (psoriasis, pityriasis subia pilasis, acne, ichthyosis) and pruritus, as well as tumor diseases and precancerous stages (for example, tumors of the intestines, carcinomas of the breast, lung tumors, prostate carcinomas, leukemias, T-cell lymphomas, melanomas, Betazell carcinoma, squamous carcinoma, actinic keratoses, cervix dysplasias, and metastasizing tumors of any type).

Also, for the treatment and prophylaxis of diseases that are characterized by a disequilibrium of the immune system, the substances according to the invention are suitable. These include eczemas and diseases of the atopic Formon series and inflammatory diseases (rheumatoid arthritis, respiratory tract diseases, e.g., asthma, Crohn's disease), as well as auto-immune diseases, such as, for example, multiple sclerosis, diabetes mellitus type I, myasthenia gravis, lupus erythematosus, scleroderma, bullous skin diseases (pemphigus, pemphigoid), also rejection reactions in the case of autologous, allogeneic or xenogeneic transplants, as well as AIDS. In all of these diseases, the new compounds of general formula I can also be combined advantageously with other substances that have an immunosuppressive action, such as cyclosporin A, FK 506, rapamycin and anti-CD 4-antibodies.

The substances are also suitable for therapy of secondary hyperparathyroidism and renal osteodystrophia because of the property of calcitriols to drop the parathormone synthesis.

Owing to the presence of the vitamin D receptor in the insulin-producing cells of the pancreas, the substances are suitable for the therapy of diabetes mellitus type II by increasing the insulin secretion.

In cell populations of the hair follicle, which contribute decisively to hair growth or to hair cycle regulation, it was possible to detect vitamin D₃ receptor proteins [W. E. Stumpf et al., Cell Tissue Res. 238, 489 (1984); P. Milde et al., J. Invest. Dermatol. 97, 230 (1991)]. In addition, in-vitro findings on isolated hair follicle keratinocytes show a proliferation-inhibiting and differentiation-stimulating influence of 1,25-(OH)₂D₃.

From clinical observations, it is known that the vitamin D₃-resistant rickets often accompanies alopecia, which develops in early infancy. Experimental findings show that the vitamin D₃ binding site of the VDR in this disease mutates, i.e., is defective [K. Kristjansson et al., J. Clin. Invest. 92, 12 (1993)]. Keratinocytes, which were isolated from the hair follicles of these patients, do not react in vitro to the addition of 1,25-(OH)₂D₃ [S. Arase et al., J. Dermatol. Science 2, 353 (1991)].

A decisive role of 1,25-(OH)₂D₃ in the regulation of hair growth can be deduced from these findings.

These analogs are therefore also suitable for the production of pharmaceutical agents for the treatment of diseases that accompany disrupted hair growth (androgenetic alopecia, alopecia areata/totalis, chemotherapy-induced alopecia) or for supporting physiological hair growth without causing the side effects of calcitriol (especially hypercalcemia).

Senile and postmenopausal osteoporosis is characterized by an increased bone turnover with an overall negative balance. Owing to the bone shrinkage especially of trabecular bones, fractures result to an increased extent. Owing to the stimulating action of calcitriol, both in the number and the conduct of synthesis of cells forming new bones (osteoblasts), the substances according to the invention are suitable for therapy and prophylaxis of senile and postmenopausal osteoporosis (EP 0 634 173 A1), of steroid-induced osteoporosis, as well as of accelerated healing of anthroplasties without causing the side effects of calcitriol (especially hypercalcemia). For the therapy of various forms of osteoporosis, they can be combined advantageously with estradiol or other derivatives of estrogen.

Finally, it was possible to show that calcitriol increases the synthesis of a growth substance for nerve cells (nerve growth factor) [M. S. Saporito et al. Brain Res. 633, 189 (1994)]. The compounds according to the invention are therefore also suitable for treating degenerative diseases of the peripheral and central nervous system, such as Alzheimer's disease and amyotrophic lateral sclerosis.

In addition, it has been found that certain compounds of general formula I in HL 60 cells antagonize, surprisingly enough, the action of calcitriol.

Such compounds can be used for the therapy of hypercalcemias, such as, for example, in hypervitaminosis D or intoxication with calcitriol and calcitriol-like active substances, or in the case of increased extrarenal calcitriol synthesis in granulomatous diseases (sarcoidosis, tuberculosis). Also, paraneoplastic hypercalcemias (for example, in osteolytic metastases and tumors with increased synthesis of parathormone-related peptides) as well as in hypercalcemias in the case of hyperparathyroidism.

In addition, calcitriol antagonists can be used for birth control. In the reproductive tracts of female and male animals, the vitamin D receptor is expressed. It is known that the female and male fertility of vitamin-D-deficient animals is reduced. By short-term substitution of calcitriol, the reproductive output can be increased. Calcitriol antagonists are therefore able to influence female and male fertility.

Since calcitriol, under certain conditions, shows an immunosuppressive action, calcitriol receptor antagonists can also be used as immunostimulants, e.g., in the case of weak defenses against infections, AIDS.

Calcitriol is known to be able to modulate hair growth. Calcitriol antagonists can therefore be used therapeutically in the case of undesirable hair growth, e.g., in hirsutism.

Vitamin D has long been known to play a stimulating role in the formation of arteriosclerotic plaque. In such vascular lesions, a calcitriol-regulated protein, osteopontin, is found to be increased, to which a role in vascular sclerosis is attributed [R. Eisenstein et al. Arch. Path. 77, 27 (1964), L. A. Fitzpatrick et al., J. Clin. Invest. 94, 1597 (1994)]. Calcitriol antagonists are therefore suitable for therapy and prophylaxis of all types of arteriosclerosis.

For all therapeutic applications presented, it is true that the compounds according to the invention are able to achieve a therapeutic action in the above-mentioned clinical pictures without causing the side effects of calcitriol (especially hypercalcemia).

This invention thus also relates to pharmaceutical preparations that contain at least one compound according to general formula I together with a pharmaceutically compatible vehicle.

The compounds can be formulated as solutions in pharmaceutically compatible solvents or as emulsions, suspensions or dispersions in suitable pharmaceutical solvents or vehicles or as pills, tablets or capsules, which contain solid vehicles in a way that is known in the art. For topical use, the compounds are advantageously formulated as creams or ointments or in a similar form of pharmaceutical agent that is suitable for topical use. Each such formulation can also contain other pharmaceutically compatible and nontoxic adjuvants, such as, e.g., stabilizers, antioxidants, binders, dyes, emulsifiers or flavoring additives. The compounds are advantageously administered by injection, intravenous infusion of suitable sterile solutions, as an aerosol via bronchial tubes and lungs, or as an oral dosage via the alimentary tract or topically in the form of creams, ointments, lotions or suitable transdermal patches, as is described in EP-A 0 387 077.

The daily dose is approximately

0.03 ig/kg/day−600 ig/patient/day

preferably 0.1 ig/kg/day−500 ig/kg/day.

Process for the Production of the Compounds According to the Invention

The production of the vitamin D derivatives of general formula I is carried out according to the invention from a compound of general formula II,

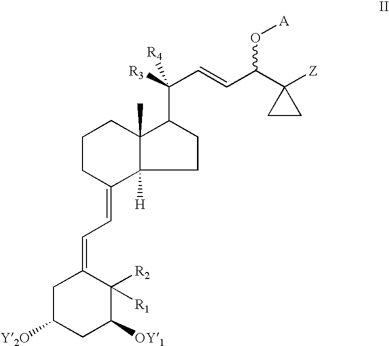

in which Y′₁ and Y′₂ mean hydroxy protective groups.

The protective groups are preferably alkyl-, aryl- or mixed alkylaryl-substituted silyl groups, e.g., the trimethylsilyl (TMS), triethylsilyl (TES), tert-butyldimethylsilyl (TBDMS), tert-butyldiphenylsilyl (TBDPS) or triisopropylsilyl (TIPS) groups or another standard hydroxy protective group (trimethyl-silylethoxymethyl, methoxymethyl, methoxyethoxymethyl, ethoxyethyl, tetrahydrofuranyl and tetrahydropyranyl groups) (see T. W. Greene, P. G. M. Wuts “Protective Groups in Organic Synthesis,” 2^{nd} Edition, John Wiley & Sons, 1991).

By simultaneous or successive cleavage of the hydroxy protective groups and optionally by partial, successive or complete esterification of the free hydroxyl groups, the compound of general formula II is converted into a compound of general formula I.

In the case of the silyl protective groups or-the trimethylsilylethoxymethyl group, tetrabutylammonium fluoride, hydrofluoric acid or hydrofluoric acid/pyridine or acidic ion exchanger is used for their cleavage. In the case of the ether groups (methoxymethyl, methoxyethoxymethyl, ethoxyethyl, tetrahydropyranyl ether) and ketals, the latter are cleaved off under catalytic action of acid, for example, p-toluenesulfonic acid, pyridinium-p-toluenesulfonate, acetic acid, hydrochloric acid, phosphoric acid or an acidic ion exchanger. The esterification of the free hydroxy groups can be carried out, if desired, according to standard processes with the corresponding carboxylic acid chlorides, -bromides or -anhydrides.

The production of the compounds of general formula II is carried out by reaction of the compounds of general formula III (WO 94/07853, WO 97/00242, WO 97/41096) with suitable acid chlorides, acid bromides or acid anhydrides in a basic medium under standard conditions.

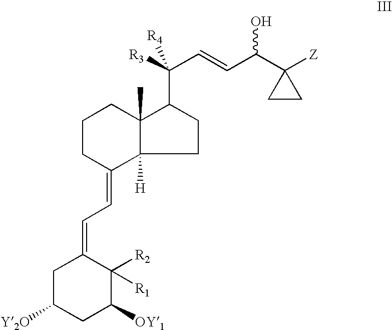

The separation of diastereomers can be carried out chromatographically in the stage of one of the above-mentioned compounds (I), (II) or (III).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The examples below are used for a more detailed explanation of the subject of the invention, without intending that it be limited to these examples.

### EXAMPLE 1

(5Z,7E)-(1S,3R,24S)-24-Acetoxy-25-(5-butyloxazole-2-yl)-26,27-cyclo-9,10-secocholesta 5,7,10(19)-triene-1,3-diol 3a and (5Z,7E)-(1S,3R,24R)-24-(acetoxy)-25-(5-butyloxazole-2-yl)-26,27-cyclo-9,10-secocholesta-5,7,10(19)-triene-1,3-diol 3b

1. 300 mg of (5Z,7E)-(1S,3R)-1,3-bis[[(1,1,-dimethylethyl)dimethylsilyl]oxy]-(5-butyloxazole-2-yl)-26,27-cyclo-9,10-secocholesta-5,7,10(19)-triene-24-ol 1 is introduced into 5 ml of dichloromethane, and 0.08 ml of triethylamine and 0.6 ml of acetic acid anhydride as well as a spatula tip full of dimethylaminopyridine are added. It is stirred for 2 hours at 25° C. and then quenched with sodium bicarbonate. It is extracted with dichloromethane, the organic phase is dried on sodium sulfate, and the solvent is removed. The residue is chromatographed on silica gel with ethyl acetate/hexane, whereby 276 mg of (5Z,7E)-(1S,3R)-24-acetoxy-3-bis[[(1,1-dimethylethyl)-dimethylsilyl]oxy]-(5-butyloxazole-2-yl)-26,27-cyclo-9,10-secocholesta-5,7,10(19)-triene 2 accumulates as a colorless foam (diastereomers in terms of C-24).

¹H NMR (CDCl₃): ä=0.08 ppm (s, 12H); 0.52 (s, 3H); 0.88 (s, 18H); 0.93 (t, 3H); 0.98 (d, 3H); 2.05 (s, 3H); 4.18 (m, 1H); 4.38 (m, 1H); 4.85 (s, 1H); 5.18 (s, 1H); 5.45 (m, 1H); 5.50 (d, 1H); 5.61 (m, 1H); 6.00 (d, 1H); 6.23 (d, 1H); 6.60/6.53 (s, 1H)

2. 140 mg of bis-silyl ether 2 is dissolved in 7 ml of tetrahydrofuran, 0.8 ml of hydrogen fluoride-pyridine complex is added, and it is stirred for 3 hours at 25° C. The reaction mixture is carefully poured onto sodium bicarbonate solution, and then it is extracted with ethyl acetate. The organic phase is washed with sodium chloride solution and dried on sodium sulfate. After the solvent evaporates, the residue is chromatographed on silica gel with ethyl acetate/hexane, whereby 70 mg of the diastereomer mixture of 3a and 3b is obtained, which is separated by HPLC into title compounds 3a (22 mg) and 3b (19 mg), which accumulate as colorless foams.

¹H-NMR (CD₂Cl₂): 3a: ä=0.51 ppm (s, 3H); 0.89 (t, 3H); 0.97 (d, 3H); 1.97 (s, 3H); 2.57 (t, 2H); 4.16 (m, 1H); 4.37 (m, 1H); 4.94 (s, 1H); 5.27 (s, 1H); 5.37 (dd, 1H); 5.52 (d, 1H); 5.58 (dd, 1H); 5.98 (d, 1H); 6.32 (d, 1H); 6.52 (s, 1H)

3b: ä=0.51 ppm (s, 3H); 0.90 (t, 3H); 0.98 (d, 3H); 1.98 (s, 3H); 2.57 (t, 2H); 4.16 (m, 1H); 4.37 (m, 1H); 4.94 (s, 1H); 5.28 (s, 1H); 5.38 (dd, 1H); 5.50 (d, 1H); 5.57 (dd, 1H); 5.99 (d, 1H); 6.34 (d, 1H); 6.55 (s, 1H)

### EXAMPLE 2

### (5Z,7E)-(1S,3R,24S)-25-(5-Butyloxazole-2-yl)-24-(2,2-dimethyl-1-oxopropyl)-26,27-cyclo-9,10-secocholesta-5,7,10(19)-triene-1,3-diol 5a and (5Z,7E)-(1S,3R,24R)-25-(5-butyloxazole-2-yl)-24-(2,2-dimethyl-1-oxopropyl)-26,27-cyclo-9,10-secocholesta-5,7,10(19)-triene-1,3-diol 5b

3. 3.59 g of (5Z,7E)-(1S,3R)-1,3-bis[[(1,1-dimethylethyl)dimethylsilyl]oxy]-(5-butyloxazole-2-yl)-26,27-cyclo-9,10-secocholesta-5,7,10(19)-triene-24-ol 1 is introduced into 70 ml of pyridine, and 1.72 ml of pivalic acid chloride as well as a spatula tip full of dimethylaminopyridine are added. It is stirred for 24 hours at 25° C. and then quenched with sodium bicarbonate. It is extracted with ethyl acetate, the organic phase is dried on sodium sulfate, and the solvent is removed. The residue is chromatographed on silica gel with ethyl acetate/hexane, whereby 3.38 g of (5Z,7E)-(1S,3R)-1,3-bis[[(1,1-dimethylethyl)dimethylsilyl]oxy]-(5-butyl-oxazole-2-yl)-24-(2,2-dimethyl-1-oxopropyl)-26,27-cyclo-9,10-secocholesta-5,7,10(19)-triene 4 accumulates as a colorless foam (diastereomers in terms of C-24).

¹H-NMR (CDCl₃): ä=0.08 ppm (s, 12H); 0.52 (s, 3H); 0.89 (s, 18H); 0.93 (t, 3H); 0.98/0.99 (d, 3H); 1.18 (s, 9H); 4.19 (m, 1H); 4.38 (m, 1H); 4.85 (s, 1H); 5.18 (s, 1H); 5.47 (m, 1H); 5.51/5.52 (d, 1H); 5.60 (m, 1H); 5.99 (d, 1H); 6.22 (d, 1H); 6.58/6.53 (s, 1H)

4. 3.19 g of bis-silyl ether 4 is dissolved in 105 ml of tetrahydrofuran, 7.6 ml of hydrogen fluoride-pyridine complex is added, and it is stirred for 1 hour at 25° C. The reaction mixture is carefully poured onto sodium bicarbonate solution and then is extracted with ethyl acetate. The organic phase is washed with sodium chloride solution and dried on sodium sulfate. After the solvent evaporates, the residue is chromatographed on silica gel with ethyl acetate/hexane, whereby 1.73 g of the diastereomer mixture of 5a and 5b is obtained, which is separated by HPLC into title compounds 5a (780 mg) and 5b (823 mg), which accumulate as colorless foams.

¹H-NMR (CD₂Cl₂): 5a: ä=0.50 ppm (s, 3H); 0.90 (t, 3H); 0.98 (d, 3H); 1.14 (s, 9H); 2.57 (t, 2H); 4.16 (m, 1H); 4.37 (m, 1H); 4.94 (s, 1H); 5.27 (s, 1H); 5.34 (dd, 1H); 5.50 (d, 1H); 5.58 (dd, 1H); 5.99 (d, 1H); 6.34 (d, 1H); 6.52 (s, 1H)

5b: ä=0.51 ppm (s, 3H); 0.91 (t, 3H); 0.98 (d, 3H); 1.13 (s, 3H); 2.57 (t, 2H); 4.16 (m, 1H); 4.37 (m, 1H); 4.94 (s, 1H); 5.28 (s, 1H); 5.37 (dd, 1H); 5.49 (d, 1H); 5.58 (dd, 1H); 5.99 (d, 1H); 6.34 (d, 1H); 6.54 (s, 1H)

### EXAMPLE 3

### (5Z,7E)-(1S,3R,24S)-25-(5-Butyloxazole-2-yl)-24-(1-oxobutyl)-26,27-cyclo-9,10-secocholesta-5,7,10(19)-triene-1,3-diol 7a and (5Z,7E)-(1S,3R,24R)-25-(5-butyloxazole-2-yl)-24-(1-oxobutyl)-26,27-cyclo-9,10-secocholesta-5,7,10(19)-triene-1,3-diol 7b

5. 500 mg of (5Z,7E)-(1S,3R)-1,3-bis[[(1,1-dimethyl-ethyl)dimethylsilyl]oxy]-(5-butyloxazole-2-yl)-26,27-cyclo-9,10-secocholesta-5,7,10(19)-triene-24-ol 1 is introduced into 10 ml of pyridine, and 0.2 ml of butyric acid chloride as well as a spatula tip full of dimethylaminopyridine are added. It is stirred for 8 hours at 25° C. and then quenched with sodium bicarbonate. It is extracted with ethyl acetate, the organic phase is dried on sodium sulfate, and the solvent is removed. The residue is chromatographed on silica gel with ethyl acetate/hexane, whereby 383 mg of (5Z,7E)-(1S,3R)-1,3-bis[[(1,1-dimethylethyl)dimethylsilyl]oxy]-(5-butyloxazole-2-yl)-24-(1-oxobutyl)-26,27-cyclo-9,10-secocholesta-5,7,10(19)-triene 6 accumulates as a colorless foam (diastereomers in terms of C-24).

¹H-NMR (CDCl₃): ä=0.08 ppm (s, 12H); 0.52 (s, 3H); 0.89 (s, 18H); 0.92 (t, 3H); 0.93 (t, 3H); 0.98/0.99 (d, 3H); 4.19 (m, 1H); 4.38 (m, 1H); 4.85 (s, 1H); 5.18 (s, 1H); 5.40 (m, 1H); 5.51/5.52 (d, 1H); 5.59 (m, 1H); 5.99 (d, 1H); 6.22 (d, 1H); 6.57 (s, 1H)

6. 3.19 g of bis-silyl ether 6 is dissolved in 10 ml of tetrahydrofuran, 1 ml of hydrogen fluoride-pyridine complex is added, and it is stirred for 6 hours at 25° C. The reaction mixture is carefully poured onto sodium bicarbonate solution and then is extracted with ethyl acetate. The organic phase is washed with sodium chloride solution and dried on sodium sulfate. After the solvent evaporates, the residue is chromatographed on silica gel with ethyl acetate/hexane, whereby 204 mg of the diastereomer mixture of 7a and 7b is obtained, which is separated by HPLC into title compounds 7a (78 mg) and 7b (89 mg), which accumulate as colorless foams.

¹H-NMR (CD₂Cl₂): 7a: ä=0.52 ppm (s, 3H); 0.90 (t, 3H); 0.91 (t, 3H); 0.98 (d, 3H); 2.23 (t, 2H); 2.58 (t, 2H); 4.16 (m, 1H); 4.38 (m, 1H); 4.93 (s, 1H); 5.29 (s, 1H); 5.36 (dd, 1H); 5.57 (d, 1H); 5.58 (dd, 1H); 6.00 (d, 1H); 6.36 (d, 1H); 6.53 (s, 1H)

7b: ä0.52 ppm (s, 3H); 0.90 (t, 3H); 0.91 (t, 3H); 0.99 (d, 3H); 2.23 (t, 2H); 2.58 (t, 2H); 4.17 (m, 1H); 4.38 (m, 1H); 4.94 (s, 1H); 5.29 (s, 1H); 5.39 (dd, 1H); 5.54 (d, 1H); 5.59 (dd, 1H); 6.00 (d, 1H); 6.35 (d, 1H); 6.53 (s, 1H)

### EXAMPLE 4

### (5Z,7E)-(1S,3R,24S)-25-(5-Butyloxazole-2-yl)-24-(2-methyl-1-oxopropyl)-26,27-cyclo-9,10-secocholesta-5,7,10(19)-triene-1,3-diol 9a and (5Z,7E)-(1S,3R,24R)-25-(5-butyloxazole-2-yl)-24-(2-methyl-1-oxopropyl)-26,27-cyclo-9,10-secocholesta-5,7,10(19)-triene-1,3-diol 9b

7. 500 mg of (5Z,7E)-(1S,3R)-1,3-bis[[(1,1-dimethyl-ethyl)dimethylsilyl]oxy]-(5-butyloxazole-2-yl)-26,27-cyclo-9,10-secocholesta-5,7,10(19)-triene-24-ol 1 is introduced into 10 ml of pyridine, and 0.2 ml of i-butyric acid chloride as well as a spatula tip full of dimethylaminopyridine are added. It is stirred for 3 hours at 25° C. and then quenched with sodium bicarbonate. It is extracted with ethyl acetate, the organic phase is dried on sodium sulfate, and the solvent is removed. The residue is chromatographed on silica gel with ethyl acetate/hexane, whereby 375 mg of (5Z,7E)-(1S,3R)-1,3-bis[[(1,1-dimethylethyl)dimethylsilyl]oxy]-(5-butyloxazole-2-yl)-24-(2-methyl-1-oxopropyl)-26,27-cyclo-9,10-secocholesta-5,7,10(19)-triene 8 accumulates as a colorless foam (diastereomers in terms of C-24).

¹H-NMR (CDCl₃): ä=0.08 ppm (s, 12H); 0.52 (s, 3H); 0.89 (s, 18H); 0.92 (t, 3H; 0.98/0.99 (d, 3H); 1.14 (d, 6H); 4.19 (m, 1H); 4.38 (m, 1H); 4.85 (s, 1H); 5.18 (s, 1H); 5.40 (m, 1H); 5.52/5.53 (d, 1H); 5.60 (m, 1H); 6.00 (d, 1H); 6.23 (d, 1H); 6.58 (s, 1H)

8. 881 mg of bis-silyl ether 8 is dissolved in 10 ml of tetrahydrofuran, 1 ml of hydrogen fluoride-pyridine complex is added, and it is stirred for 6 hours at 25° C. The reaction mixture is carefully poured onto sodium bicarbonate solution and then is extracted with ethyl acetate. The organic phase is washed with sodium chloride solution and dried on sodium sulfate. After the solvent evaporates, the residue is chromatographed on silica gel with ethyl acetate/hexane, whereby 231 mg of the diastereomer mixture of 9a and 9b is obtained, which is separated by HPLC into title compounds 9a (81 mg) and 9b (60 mg), which accumulate as colorless foams.

¹H-NMR (CD₂Cl₂): 9a: ä=0.53 ppm (s, 3H); 0.90 (t, 3H); 0.98 (d, 3H); 1.09 (233 d, 6H); 2.49 (hept, 1H); 2.58 (t, 2H); 4.17 (m, 1H); 4.38 (m, 1H); 4.94 (s, 1H); 5.29 (s, 1H); 5.36 (dd, 1H); 5.55 (d, 1H); 5.58 (dd, 1H); 5.99 (d, 1H); 6.35 (d, 1H); 6.53 (s, 1H)

9b: ä=0.53 ppm (s, 3H); 0.91 (t, 3H); 0.99 (d, 3H); 1.09 (2×d, 6H); 2.50 (hept, 1H); 2.58 (t, 2H); 4.17 (m, 1H); 4.38 (m, 1H); 4.95 (s, 1H); 5.29 (s, 1H); 5.38 (dd, 1H); 5.55 (d, 1H); 5.59 (dd, 1H); 6.00 (d, 1H); 6.35 (d, 1H); 6.53 (s, 1H)

### EXAMPLE 5a

### (5Z,7E)-(1S,3R,24S)-25-(5-Butyloxazole-2-yl)-24-[[(methyl-amino)carbonyl]oxy]-26,27-cyclo-9,10-secocholesta-5,7,10(19)-triene-1,3-diol 11a

b 9. 150 mg of (5Z,7E)-(1S,3R,24S)-1,3-bis[[(1,1-dimethylethyl)dimethylsilyl]oxy]-(5-butyloxazole-2-yl)-26,27-cyclo-9,10-secocholesta-5,7,10(19)-triene-24-ol 1a is introduced into 5 ml of dimethylformamide, and 105 mg of methyl isocyanate is added. It is stirred for 1 day at 25° C. and then quenched with sodium chloride solution. It is extracted with ethyl acetate, the organic phase is dried on sodium sulfate, and the solvent is removed. The residue is chromatographed on silica gel with ethyl acetate/hexane, whereby 136 mg of (5Z,7E)-(1S,3R,24S)-1,3-bis[[(1,1-dimethylethyl)-dimethylsilyl]oxy]-(5-butyloxazole-2-yl)-24-[[(methylamino)carbonyl]oxy]-26,27-cyclo-9,10-secocholesta-5,7,10(19)-triene 10a accumulates as a colorless foam.

¹H-NMR (CDCl₃): ä=0.08 ppm (s, 12H); 0.52 (s, 3H); 0.88 (s, 18H); 0.93 (t, 3H); 0.99 (d, 3H); 2.78 (d, 6H); 4.18 (m, 1H); 4.36 (m, 1H); 4.66 (m, 1H); 4.86 (s, 1H); 5.17 (s, 1H); 5.28 (m, 1H); 5.45 (m, 1H); 5.48 (d, 1H); 5.61 (m, 1H); 6.01 (d, 1H); 6.23 (d, 1H); 6.5 (s, 1H)

10. 128 mg of bis-silyl ether 10a is dissolved in 8 ml of tetrahydrofuran, 332 mg of tetrabutylammonium fluoride hydrate is added, and it is stirred for 1 day at 25° C. The reaction mixture is carefully poured onto sodium chloride solution, extracted with ethyl acetate, and dried on sodium sulfate. After the solvent evaporates, the residue is chromatographed on silica gel with ethyl acetate/hexane, whereby 52 mg of 11a is obtained as a colorless foam.

¹H-NMR (CD₂Cl₂): ä=0.54 ppm (s, 3H); 0.93 (t, 3H); 0.99 (d, 3H); 1.97 (s, 3H); 2.28 (m, 1H); 2.58 (t, 2H); 2.74 (d, 6H); 4.18 (m, 1H); 4.40 (m, 1H); 4.79 (m, 1H); 4.94 (s,1H); 5.30 (s, 1H); 5.33 (m, 1H); 5.37 (dd, 1H); 5.43 (d, 1H); 5.62 (dd, 1H); 6.02 (d, 1H); 6.37 (d, 1H); 6.58 (s, 1H)

### EXAMPLE 5b

### (5Z,7E)-(1S,3R,24R)-25-(5-Butyloxazole-2-yl)-24-[[(methylamino)-carbonyl]oxy]-26,27-cyclo-9,10-secocholesta-5,7,10(19)-triene-1,3-diol 11b

11. 150 mg of (5Z,7E)-(1S,3R,24R)-1,3-bis[[(1,1-dimethylethyl)dimethylsilyl]oxy]-(5-butyloxazole-2-yl)-26,27-cyclo-9,10-secocholesta-5,7,10(19)-triene-24-ol 1b is introduced into 5 ml of dimethylformamide, and 105 mg of methyl isocyanate is added. It is stirred for 2 days at 25° C. and then quenched with sodium chloride solution. It is extracted with ethyl acetate, the organic phase is dried on sodium sulfate, and the solvent is removed. The residue is chromatographed on silica gel with ethyl acetate/hexane, whereby 125 mg of (5Z,7E)-(1S,3R,24R)-1,3-bis[[(1,1-dimethylethyl)dimethylsilyl]oxy]-(5-butyl-oxazole-2-yl)-24-[[(methylamino)carbonyl]oxy]-26,27-cyclo-9,10-secocholesta-5,7,10(19)-triene 10b accumulates as a colorless foam.

¹H-NMR (CDCl₃): ä=0.08 ppm (s, 12H); 0.52 (s, 3H); 0.89 (s, 18H); 0.93 (t, 3H); 1.01 (d, 3H); 2.78 (d, 6H); 4.19 (m, 1H); 4.37 (m, 1H); 4.70 (m, 1H); 4.86 (s, 1H); 5.10 (s, 1H); 5.23 (m, 1H); 5.47-5.65 (m, 2H); 6.00 (d, 1H); 6.23 (d, 1H); 6.58 (s, 1H).

12. 140 mg of bis-silyl ether 10b is dissolved in 8 ml of tetrahydrofuran, 332 mg of tetrabutylammonium fluoride hydrate is added, and it is stirred for 3 days at 25° C. The reaction mixture is carefully poured onto sodium chloride solution, extracted with ethyl acetate and dried on sodium sulfate. After the solvent evaporates, the residue is chromatographed on silica gel with ethyl acetate/hexane, whereby 42 mg of 11b is obtained as a colorless foam.

¹H-NMR (CD₂Cl₂): ä=0.54 ppm (s, 3H); 0.93 (t, 3H); 1.02 (d, 3H); 2.58 (t, 2H); 2.74 (d, 6H); 4.17 (m, 1H); 4.38 (m, 1H); 4.67 (m, 1H); 4.96 (s, 1H); 5.10 (s, 1H); 5.28 (s, 1H); 5.44 (dd, 1H); 5.53 (d, 1H); 5.58 (dd, 1H); 6.18 (d, 1H); 6.37 (d, 1H); 6.53 (s, 1H).

### EXAMPLE 6

### (5Z,7E)-(1S,3R,24R)-25-(5-Butyloxazole-2-yl)-24-[[(ethylamino)carbonyl]oxy]-26,27-cyclo-9,10-secocholesta-5,7,10(19)-triene-1,3-diol 13b

13. 150 mg of (5Z,7E)-(1S,3R,24R)-1,3-bis[[(1,1-dimethylethyl)dimethylsilyl]oxy]-(5-butyloxazole-2-yl)-26,27-cyclo-9,10-secocholesta-5,7,10(19)-triene-24-ol 1b is introduced into 5 ml of dimethylformamide, and 0.124 ml of ethyl isocyanate is added. It is stirred for 3 days at 25° C. and then quenched with sodium chloride solution. It is extracted with ethyl acetate, the organic phase is dried on sodium sulfate, and the solvent is removed. The residue is chromatographed on silica gel with ethyl acetate/hexane, whereby 140 mg of (5Z,7E)-(1S,3R,24R)-1,3-bis[[(1,1-dimethylethyl)dimethylsilyl]oxy]-(5-butyl-oxazole-2-yl)-24-[[(ethylamino)carbonyl]oxy]-26,27-cyclo-9,10-secocholesta-5,7,10(19)-triene 12b accumulates as a colorless foam.

¹H-NMR (CDCl₃): ä=0.06 ppm (s, 12H); 0.51 (s, 3H); 0.88 (s, 18H); 0.93 (t, 3H); 1.02 (d, 3H); 1.12 (t, 3H); 4.18 (m, 1H); 4.38 (m, 1H); 4.72 (m, 1H); 4.86 (s, 1H); 5.18 (s, 1H); 5.23 (m, 1H); 5.45-5.60 (m, 2H); 5.99 (d, 1H); 6.22 (d, 1H); 6.58 (s, 1H)

14. 65 mg of bis-silyl ether 12b is dissolved in 5 ml of tetrahydrofuran, 172 mg of tetrabutylammonium fluoride hydrate is added, and it is stirred for 3 days at 25° C. The reaction mixture is carefully poured onto sodium chloride solution, extracted with ethyl acetate and dried on sodium sulfate. After the solvent evaporates, the residue is chromatographed on silica gel with ethyl acetate/hexane, whereby 25 mg of 13b is obtained as a colorless foam.

¹H-NMR (CD₂Cl₂): ä=0.55 ppm (s, 3H); 0.92 (t, 3H); 1.02 (d, 3H); 1.11 (t, 3H); 2.59 (t, 2H); 2.83 (m, 1H); 3.17 (m, 2H); 4.17 (m, 1H); 4.38 (m, 1H); 4.80 (m, 1H); 4.95 (s, 1H); 5.28-5.38 (m, 2H); 5.52 (d, 1H); 5.58 (dd, 1H); 6.01 (d, 1H); 6.36 (d, 1H); 6.58 (s, 1H)

### EXAMPLE 7

### (5Z,7E)-(1S,3R,24R)-25-(5-Butyloxazole-2-yl)-24-[[((pentafluorophenyl)amino)-carbonyl]oxy]-26,27-cyclo-9,10-secocholesta-5,7,10(19)-triene-1,3-diol 15b

15. 30 mg of (5Z,7E)-(1S,3R,24R)-1,3-bis[[(1,1-dimethylethyl)-dimethylsilyl]oxy]-(5-butyloxazole-2-yl)-26,27-cyclo-9,10-secocholesta-5,7,10(19)-triene-24-ol 1b is introduced into 2 ml of tetrahydrofuran, and 0.068 ml of pentafluorophenylisocyanate is added. It is stirred for 1 day at 25° C. and then quenched with sodium chloride solution. It is extracted with ethyl acetate, the organic phase is dried on sodium sulfate, and the solvent is removed. The residue is chromatographed on silica gel with ethyl acetate/hexane, whereby 61 mg of (5Z,7E)-(1S,3R,24R)-1,3-bis[[(1,1-dimethylethyl)dimethylsilyl]oxy]-(5-butyloxazole-2-yl)-24-[[((pentafluorophenyl)amino)carbonyl]oxy]-26,27-cyclo-9,10-secocholesta-5,7,10(19)-triene 14b accumulates as a colorless foam.

¹H-NMR (CDCl₃): ä=0.01 ppm (s, 12H); 0.47 (s, 3H); 0.85 (s, 18H); 0.90 (t, 3H); 0.96 (d, 3H); 2.55 (m, 2H); 4.14 (m, 1H); 4.31 (m, 1H); 4.80 (s, 1H); 5.14 (s, 1H); 5.19 (m, 1H); 5.45 (m, 1H); 5.61 (m, 1H); 5.92 (d, 1H); 6.17 (d, 1H); 6.65 (s, 1H)

16. 30 mg of bis-silyl ether 14b is dissolved in 2 ml of tetrahydrofuran, 68 mg of tetrabutylammonium fluoride hydrate is added, and it is stirred for 1 day at 25° C. The reaction mixture is carefully poured onto sodium chloride solution, extracted with ethyl acetate and dried on sodium sulfate. After the solvent evaporates, the residue is chromatographed on silica gel with ethyl acetate/hexane, whereby 11 mg of 15b is obtained as a colorless foam.

¹H-NMR (CDCl₃): ä=0.53 ppm (s, 3H); 0.91 (t, 3H); 1.03 (d, 3H); 2.56 (t, 2H); 4.23 (m, 1H); 4.47 (m, 1H); 4.96 (s, 1H); 5.13 (d, 1H); 5.37 (s, 1H); 5.50 (dd, 1H); 5.62 (d, 1H); 5.99 (dd, 1H); 6.33 (d, 1H); 6.32 (d, 1H); 6.62 (s, 1H)

### EXAMPLE 8a

### (5Z,7E)-(1S,3R,24S)-25-(5-Butyloxazole-2-yl)-24-[[(2,2-dimethyl-3-hydroxy)-1-oxopropyl]oxy]-26,27-cyclo-9,10-secocholesta-5,7,10(19)-triene-1,3-diol 17a

17. 351 mg of (5Z,7E)-(1S,3R)-1,3-bis[[(1,1-dimethylethyl)dimethylsilyl]oxy]-(5-butyloxazole-2-yl)-26,27-cyclo-9,10-secocholesta-5,7,10(19)-triene-24-ol 1a is introduced into 15 ml of dichloromethane, and 0.23 ml of triethylamine and 265 mg of 2,2-dimethyl-3-[[(1,1-dimethylethyl)dimethylsilyl]oxy]-2,2-dimethylpropanoic acid chloride 21 as well as a spatula tip full of DMAP are added. It is stirred for 1 day at 25° C. The reaction mixture is carefully poured onto sodium bicarbonate solution and then is extracted with ethyl acetate. The organic phase is washed with sodium chloride solution and dried on sodium sulfate. After the solvent evaporates, the residue is chromatographed on silica gel with ethyl acetate/hexane, whereby 222 mg of (5Z,7E)-(1S,3R,24S)-1,3-bis[[(1,1-dimethylethyl)-dimethylsilyl]oxy]-(5-butyloxazole-2-yl)-24-[[2,2-dimethyl-3-[[(1,1-dimethyl)dimethylsilyl]oxy]-1-oxopropyl]oxy]-26,27-cyclo-9,10-secocholesta-5,7,10(19)-triene 16a accumulates as a colorless foam.

¹H-NMR (CDCl₃): ä=0.03 ppm (s, 18H); 0.48 (s, 3H); 0.83 (s, 27H); 0.93 (t, 3H); 1.08 (d, 3H); 1.17 (s, 6H); 3.53-3.59 (m, 2H); 4.15 (m, 1H); 4.34 (m, 1H); 4.81 (s, 1H); 5.14 (s, 1H); 5.45 (m, 1H); 5.35 (m, 1H); 5.45 (m, 1H); 5.56 (m, 1H); 5.96 (d, 1H); 6.19 (d, 1H); 6.53 (s, 1H)

18. 98 mg of bis-silyl ether 16a is dissolved in 8 ml of tetrahydrofuran, 384 mg of tetrabutylammonium fluoride hydrate is added, and it is stirred for 1 day at 25° C. The reaction mixture is carefully poured onto sodium chloride solution, extracted with ethyl acetate and dried on sodium sulfate. After the solvent evaporates, the residue is chromatographed on silica gel with ethyl acetate/hexane, whereby 11 mg of 17a is obtained as a colorless foam.

¹H-NMR (CD₂Cl₂): ä=0.53 ppm (s, 3H); 0.93 (t, 3H); 1.00 (d, 3H); 1.12 (s, 3H); 1.23 (s, 3H); 2.57 (t, 2H); 3.41 (d, 1H); 3.72 (d, 2H); 4.25 (m, 1H); 4.44 (m, 1H); 5.00 (s, 1H); 5.38-5.48 (m, 3H); 5.60 (dd, 1H); 6.01 (d, 1H); 6.47 (d, 1H); 6.61 (s, 1H)

### EXAMPLE 8b

### (5Z,7E)-(1S,3R,24R)-25-(5-Butyloxazole-2-yl)-24-[[(2,2-dimethyl-3-hydroxy)-1-oxopropyl]oxy]-26,27-cyclo-9,10-secocholesta-5,7,10(19)-triene-1,3-diol 17b

19. 200 mg of (5Z,7E)-(1S,3R)-1,3-bis[[(1,1-dimethylethyl)dimethylsilyl]oxy]-(5-butyloxazole-2yl)-26,27-cyclo-9,10-secocholesta-5,7,10(19)-triene-24-ol 1b is introduced into 8 ml of dichloromethane, and 0.13 ml of triethylamine and 150 mg of 2,2-dimethyl-3-[[(1,1-dimethylethyl)dimethylsilyl]oxy]-2,2-dimethylpropanoic acid chloride 21 as well as a spatula tip full of DMAP are added. It is stirred for 1 day at 25° C. The reaction mixture is carefully poured onto sodium bicarbonate solution and then is extracted with ethyl acetate. The organic phase is washed with sodium chloride solution and dried on sodium sulfate. After the solvent evaporates, the residue is chromatographed on silica gel with ethyl acetate/hexane, whereby 144 mg of (5Z,7E)-(1S,3R,24R)-1,3-bis[[(1,1-dimethylethyl)-dimethylsilyl]oxy]-(5-butyloxazole-2-yl)24-[[2,2-dimethyl-3-[[(1,1-dimethyl)dimethyl-silyl]oxy]-1-oxopropyl]oxy]-26,27-cyclo-9,10-secocholesta-5,7,10(19)-triene 16b accumulates as a colorless foam.

¹H-NMR (CDCl₃): ä=0.03 ppm (s, 18H); 0.51 (s, 3H); 0.85 (s, 27H); 0.93 (t, 3H); 1.03 (d, 3H); 1.17 (s, 6H); 3.53-3.59 (m, 2H); 4.20 (m, 1H); 4.38 (m, 1H); 4.85 (s, 1H); 5.17 (s, 1H); 5.32-5.65 (m, 3H); 5.99 (d, 1H); 6.23 (d, 1H); 6.56 (s, 1H)

20. 144 mg of bis-silyl ether 16b is dissolved in 7 ml of tetrahydrofuran, 321 mg of tetrabutylammonium fluoride hydrate is added, and it is stirred for 1 day at 25° C. The reaction mixture is carefully poured onto sodium chloride solution, extracted with ethyl acetate and dried on sodium sulfate. After the solvent evaporates, the residue is chromatographed on silica gel with ethyl acetate/hexane, whereby 14 mg of 17b is obtained as a colorless foam.

¹H-NMR (CDCl₃): ä=0.48 ppm (s, 3H); 0.85 (t, 3H); 0.92 (d, 3H); 1.07 (s, 3H); 1.18 (s, 3H); 2.53 (t, 2H); 3.34 (d, 1H); 3.65 (d, 2H); 4.15 (m, 1H); 4.35 (m, 1H); 4.93 (s, 1H); 5.15-5.32 (m, 3H); 5.52 (dd, 1H); 5.93 (d, 1H); 6.31 (d, 1H); 6.53 (s, 1H)

Reagent for Examples 8a and 8b

21. 9.65 ml of 2,2-dimethyl-3-hydroxypropionic acid methyl ester 18 is introduced into 200 ml of dimethylformamide, and 10.28 g of imidazole as well as, carefully, 13.68 g of tert-butyldimethylsilyl chloride are added. It is stirred for 3 days at 25° C. The reaction mixture is carefully added to sodium chloride solution and then extracted with ethyl acetate. The organic phase is washed with sodium chloride solution and dried on sodium sulfate. After the solvent evaporates, the residue is chromatographed on silica gel with ethyl acetate/hexane, whereby 16.85 g of 2,2-dimethyl-3-[[(1,1-dimethylethyl)-dimethylsilyl]oxy]-2,2-dimethylpropanoic acid methyl ester 19 accumulates as a colorless liquid.

22. 2.0 g of ester 19 is introduced into 60 ml of methanol and 20 ml of VE-water, and 1.94 g of lithium hydroxide is added. It is stirred for 1 day at 25° C. and then carefully set at pH 1 with 1N hydrochloric acid. It is extracted with ethyl acetate, and the organic phase is washed with sodium chloride solution and dried on sodium sulfate. The crude product is again dried for 15 minutes by the oil pump in a vacuum, and 1.78 g of 2,2-dimethyl-3-[[(1,1-dimethylethyl)-dimethylsilyl]oxy]-2,2-dimethylpropanoic acid 20 is obtained.

22. 1.0 g of acid 20 is introduced into 10 ml of dichloromethane, and 6.74 ml of oxalyl chloride is carefully added. It is stirred for 1 day at 25° C., and then the solvent is removed. The crude product is again dried for 15 minutes by the oil pump in a vacuum, and 1.01 g of 2,2-dimethyl-3-[[(1,1-dimethylethyl)dimethylsilyl]oxy]-2,2-dimethylpropanoic acid chloride 21 is obtained as a colorless liquid.

### EXAMPLE 9

### (5Z,7E,22E)-(1S,3R,24R)-25-(5-Butyloxazole-2-yl)-26,27-cyclo-9,10-secocholesta-5,7,10(19),22-tetraene-1,3,24-triol tris-isonicotinate 23 b

23. 20 mg of (5Z,7E,22E)-(1S,3R,24R)-25-(5-butyloxazole-2-yl)-26,27-cyclo-9,10-secocholesta-5,7,10(19),22-tetraene-1,3,24-triol 22b (WO97/41096) is introduced into 2 ml of pyridine, and 59.8 mg of isonictinoyl chloride hydrochloride is added. It is stirred for 1 day at 25° C., and then the reaction mixture is carefully poured onto sodium bicarbonate solution. It is extracted with ethyl acetate, and the organic phase is washed with sodium chloride solution. After the solvent evaporates, the residue is chromatographed on silica gel with ethyl acetate/hexane, whereby 27 mg of 23b accumulates as a colorless foam.

¹H-NMR (CD₂Cl₂): ä=0.28 ppm (s, 3H); 0.87 (t, 3H); 0.97 (d, 3H); 2.55 (t, 2H); 5.14 (s, 1H); 5.43-5.52 (m, 2H); 5.61-5.73 (m, 2H); 5.78 (t, 1H); 5.91 (d, 1H); 6.48 (d, 1H); 6.55 (s, 1H); 7.80 (m, 6H); 8.73 (m, 6H)

### EXAMPLE 10

### (5Z,7E,22E)-(1S,3R,24R)-25-(5-Butyloxazole-2-yl)-26,27-cyclo-9,10-secocholesta-5,7,10(19),22-tetraene-1,3,24-triol tribenzoate 24b

24. 20 mg of (5Z,7E,22E)-(1S,3R,24R)-25-(5-butyloxazole-2-yl)-26,27-cyclo-9,10-secocholesta-5,7,10(19),22-tetraene-1,3,24-triol 22b is introduced into 2 ml of pyridine, and 0.039 ml of benzoyl chloride is added. It is stirred for 1 day at 25° C., and then the reaction mixture is carefully poured onto sodium bicarbonate solution. It is extracted with ethyl acetate, and the organic phase is washed with sodium chloride solution. After the solvent evaporates, the residue is chromatographed on silica gel with ethyl acetate/hexane, whereby 25 mg of 24b accumulates as a colorless foam.

¹H-NMR (CD₂Cl₂): ä=0.30 ppm (s, 3H); 0.88 (t, 3H); 0.96 (d, 3H); 2.57 (t, 2H); 5.10 (d, 1H); 5.45-5.54 (m, 2H); 5.60-5.70 (m, 2H); 5.77 (m, 1H); 5.94 (d, 1H); 6.46 (d, 1H); 6.55 (s, 1H); 7.42 (m, 3H); 7.55 (m, 3H); 7.55 (m, 3H); 8.00 (m, 6H)

The entire disclosures of all applications, patents and publications, cited herein and of corresponding German application No. 101 56 596.8 filed Nov. 13, 2001 and U.S. Provisional Application Serial No. 60/331,386, filed Nov. 15, 2001 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Vitamin D derivatives of general formula I, 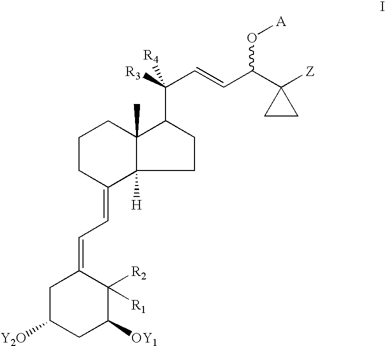
in which
R₁ and R₂ each mean a hydrogen atom or together an exocyclic methylene group,
R₃ and R₄, independently of one another, mean a hydrogen atom, a fluorine or chlorine atom, an alkyl group with 1 to 4 carbon atoms, together a methylene group or together with quaternary carbon atom 20 a 3- to 7-membered, saturated or unsaturated carbocyclic ring,
A means a group —C(X)—R⁵, —C(X)—NH—R⁵, —C(X)—N(R⁵)₂, —P(O)—(OR⁵)₂, —SO₂—OR⁵,
X stands for an oxygen atom or a sulfur atom,
R₅ means a straight-chain or branched, saturated or unsaturated alkyl chain with 1 to 10 carbon atoms, which optionally can be substituted by 1-3 hydroxy groups, a group COOR¹², a group CONR¹⁰R¹¹, a group P(O)(OR¹⁰)₂, P(O)(NR¹⁰R¹¹)₂, SO₃R¹⁰, S(O)₂NR¹⁰R¹¹, an N(R¹⁰R¹¹) group, whereby
R¹⁰ and R¹¹, independently of one another, mean hydrogen or a straight-chain or branched, saturated or unsaturated alkyl chain with 1 to 10 carbon atoms, but R¹⁰ and R¹¹ cannot simultaneously mean hydrogen,
an aromatic or heteroaromatic radical with 5-12 carbon atoms, which optionally can be substituted with nitro groups, with optionally 1-2 substituted C₁-C₆ alkyl groups, trihaloalkyl groups or alkoxy groups or halogen atoms, or an optionally substituted phenyl radical, benzyl radical or a 2-, 3- or 4-pyridyl radical,
Y₁ and Y₂, independently of one another, each mean a hydrogen atom or a group —C(O)R₆, whereby
R₆ stands for an aromatic or heteroaromatic radical with 5 to 12 C atoms, or for an aliphatic, straight-chain or branched, saturated or unsaturated C₁-C₁₂ alkyl radical, which optionally is interrupted by 1-2 oxygen atoms, 1-2 sulfur atoms and/or 1-2 NH groups and/or optionally is substituted by 1-2 hydroxy groups, 1-2 amino groups, 1-2 SH groups, 1-2 COOH groups and/or 1-2 phenyl groups,
Z means a straight-chain or branched, saturated or unsaturated 1-oxoalkyl group with 2 to up to 12 carbon atoms, a 1-oxo-(C₃-C₇)-cycloalkyl group, a benzoyl group, a 2-pyridylcarbonyl group, or a group CN, COOR₇, COSR₇, CONHR₇, CONR₇R₈, whereby
R₇ and R₈, independently of one another, stand for a hydrogen atom or a saturated or unsaturated alkyl group with 1 to 8 C atoms,
a cycloalkyl group with 3 to 8 carbon atoms,
a saturated or unsaturated alkyl group with 1 to 12 C atoms, which can have hydroxy or amino groups that are optionally substituted by A at any positions 1-3, halogen atoms or carboxylic acid esters or -amide units, and optionally is linked by a carbonyl group, a hydroxymethylene group or an ethenyldiyl unit (—CH═CH—, E— or Z-geometry) with carbon atom 25,
or a carbocyclic or heterocyclic optionally aromatic or heteroaromatic ring with 5- or 6-ring members, or a condensed ring system that consists of a 5- and a 6-membered ring or two 6-membered rings, which can be substituted by one or more halogen atoms, one or more hydroxy groups, one or more COOR₆ groups, 1-3 C₁-C₅ perfluoroalkyl groups, one or more C₁-C₅ alkyl groups, which in turn can be substituted by one or more halogen atoms, C₁-C₆ alkoxy groups and/or COOR₉ groups and/or can be interrupted by oxa, thia or aza functions, sulfoxy or sulfone groups, whereby
R₉ stands for a C₁-C₆ alkyl group, a benzyl group or a phenyl group, as well as all possible epimers or diastereomers and mixtures thereof.

2. Vitamin D derivatives according to claim 1, in which
R₁ and R₂ each mean a hydrogen atom or together an exocyclic-methylene group,
R₃ and R₄, independently of one another, mean a hydrogen atom, an alkyl group with 1 to 4 carbon atoms, together a methylene group or together with quaternary carbon atom 20 a 3-membered, saturated or unsaturated carbocyclic ring,
A means a group —C(X)—R⁵, —C(X)—NH—R⁵, or —C(X)—N(R⁵)₂,
X stands for an oxygen atom,
R₅ means a straight-chain or branched, saturated or unsaturated alkyl chain with 1 to 10 carbon atoms, which optionally can be substituted by 1-3 hydroxy groups, a group COOR¹², a group CONR¹⁰R¹¹, an N(R¹⁰R¹¹) group, whereby
R¹⁰ and R¹¹ independently of one another, mean hydrogen or a straight-chain or branched, saturated and unsaturated alkyl chain with 1 to 10 carbon atoms, but R¹⁰ and R¹¹ cannot simultaneously mean hydrogen,
an aromatic or heteroaromatic radical with 5-12 carbon atoms, which optionally can be substituted with nitro groups, with optionally 1-2 substituted C₁-C₅ alkyl groups, trihaloalkyl groups or alkoxy groups or halogen atoms, or an optionally substituted phenyl radical, benzyl radical or a 2-, 3- or 4-pyridyl radical,
Y₁ and Y₂, independently of one another, each mean a hydrogen atom or a group —C(O)R₆, whereby
R₆ stands for an aromatic or heteroaromatic radical with 5 to 12 C atoms, or for an aliphatic, straight-chain or branched, saturated or unsaturated C₁-C₁₂ alkyl radical, which optionally is interrupted by 1-2 oxygen atoms, 1-2 sulfur atoms and/or 1-2 NH groups and/or optionally is substituted by 1-2 hydroxy groups, 1-2 amino groups, 1-2 SH groups, 1-2 COOH groups and/or 1-2 phenyl groups,
Z means a straight-chain or branched, saturated or unsaturated 1-oxoalkyl group with 2 to up to 12 carbon atoms, a 1-oxo-(C₃-C₇)-cycloalkyl group, a benzoyl group, a 2-pyridylcarbonyl group, or a group CN, COOR₇, COSR₇, CONHR₇, CONR₇R₈, whereby
R₇ and R₈, independently of one another, stand for a hydrogen atom or a saturated or unsaturated alkyl group with 1 to 8 C atoms,
a cycloalkyl group with 3 to 8 carbon atoms,
a saturated or unsaturated alkyl group with 1 to 12 C atoms, which can have hydroxy or amino groups that are optionally substituted by A at any positions 1-3, halogen atoms or carboxylic acid esters or -amide units, and optionally is linked by a carbonyl group, a hydroxymethylene group or an ethenyldiyl unit (—CH═CH—, E— or Z-geometry) with carbon atom 25,
or a carbocyclic or heterocyclic optionally aromatic or heteroaromatic ring with 5- or 6-ring members, or a condensed ring system that consists of a 5- and a 6-membered ring or two 6-membered rings, which can be substituted by one or more halogen atoms, one or more hydroxy groups, one or more COOR₆ groups, 1-3 C₁-C₅ perfluoroalkyl groups, one or more C₁-C₅ alkyl groups, which in turn can be substituted by one or more halogen atoms, C₁-C₆ alkoxy groups and/or COOR₉ groups and/or can be interrupted by oxa, thia or aza functions, sulfoxy or sulfone groups, whereby
R₉ stands for a C₁-C₆ alkyl group, a benzyl group or a phenyl group, as well as all possible epimers or diastereomers and mixtures thereof.

3. Compounds according to one of the preceding claims, characterized in that R⁵ means a straight-chain or branched, saturated or unsaturated alkyl chain with 1 to 10 carbon atoms, an aromatic or heteroaromatic radical with 5-12 carbon atoms, which optionally can be substituted with 1-2 optionally substituted C₁-C₆ alkyl groups, or an optionally substituted phenyl radical, benzyl radical or a 2-, 3- or 4-pyridyl radical.

4. Compounds according to claim 4, wherein R⁵ means a branched, saturated or unsaturated alkyl chain with 3 to 10 carbon atoms, an aromatic or heteroaromatic radical with 5-12 carbon atoms, which optionally can be substituted with 1-2 optionally substituted C₁-C₆ alkyl groups, or an optionally substituted phenyl radical or a 2-, 3- or 4-pyridyl radical.

5. Compounds according to claim 1
(5Z,7E)-(1S,3R,24S)-24-Acetoxy-25-(5-butyloxazole-2-yl)-26,27-cyclo-9,10-secocholesta-5,7,10(19)-triene-1,3-diol
(5Z,7E)-(1S,3R,24R)-24-acetoxy-25-(5-butyloxazole-2-yl)-26,27-cyclo-9,10-secocholesta-5,7,10(19)-triene-1,3-diol
(5Z,7E)-(1S,3R,24S)-25-(5-butyloxazole-2-yl)-24-(2,2-dimethyl-1-oxopropoxy)-26,27-cyclo-9,10-secocholesta-5,7,10(19)-triene-1,3-diol
(5Z,7E)-(1S,3R,24R)-25-(5-butyloxazole-2-yl)-24-(2,2-dimethyl-1-oxopropoxy cyclo-9,10-secocholesta-5,7,10(19)-triene-1,3-diol
(5Z,7E)-(1S,3R,24S)-25-(5-butyloxazole-2-yl)-24-(1-oxobutoxy)-26,27-cyclo-9,10-secocholesta-5,7,10(19)-triene-1,3-diol
(5Z,7E)-(1S,3R,24R)-25-(5-butyloxazole-2-yl)-24-(1-oxobutoxy)-26,27-cyclo-9,10-secocholesta-5,7,10(19)-triene-1,3-diol
(5Z,7E)-(1S,3R,24S)-25-(5-butyloxazole-2-yl)-24-(2-methyl-1-oxobutoxy)-26,27-cyclo-9,10-secocholesta-5,7,10(19)-triene-1,3-diol
(5Z,7E)-(1S,3R,24R)-25-(5-butyloxazole-2-yl)-24-(2-methyl-1-oxobutoxy)-26,27-cyclo-9,10-secocholesta-5,7,10(19)-triene-1,3-diol
(5Z,7E)-(1S,3R,24S)-25-(5-butyloxazole-2-yl)-24-(2-methyl-1-oxopropoxy)-26,27-cyclo-9,10-secocholesta-5,7,10(19)-triene-1,3-diol
(5Z,7E)-(1S,3R,24R)-25-(5-butyloxazole-2-yl)-24-(2-methyl-1-oxopropoxy)-26,27-cyclo-9,10-secocholesta-5,7,10(19)-triene-1,3-diol
(5Z,7E)-(1S,3R,24S)-25-(5-butyloxazole-2-yl)-24-(1-oxopropoxy)-26,27-cyclo-9,10-secocholesta-5,7,10(19)-triene-1,3-diol
(5Z,7E)-(1S,3R,24R)-25-(5-butyloxazole-2-yl)-24-(1-oxopropoxy)-26,27-cyclo-9,10-secocholesta-5,7,10(19)-triene-1,3-diol
(5Z,7E)-(1S,3R,24S)-25-(5-butyloxazole-2-yl)-24-(3-hydroxy-2-methyl-1-oxopropoxy)-26,27-cyclo-9,10-secocholesta-5,7,10(19)-triene-1,3-diol
(5Z,7E)-(1S,3R,24R)-25-(5-butyloxazole-2-yl)-24-(3-hydroxy-2-methyl-1-oxopropoxy)-26,27-cyclo-9,10-secocholesta-5,7,10(19)-triene-1,3-diol
(5Z,7E)-(1S,3R,24S)-25-(5-butyloxazole-2-yl)-24-(1-oxopentoxy)-26,27-cyclo-9,10-secocholesta-5,7,10(19)-triene-1,3-diol
(5Z,7E)-(1S,3R,24R)-25-(5-butyloxazole-2-yl)-24-(1-oxopentoxy)-26,27-cyclo-9,10-secocholesta-5,7,10(19)-triene-1,3-diol
(5Z,7E)-(1S,3R,24S)-25-(5-butyloxazole-2-yl)-26,27-cyclo-24-(4-pyridylcarbonyl)-9,10-secocholesta-5,7,10(19)-triene-1,3-diol
(5Z,7E)-(1S,3R,24R)-25-(5-butyloxazole-2-yl)-26,27-cyclo-24-(4-pyridylcarbonyl)-9,10-secocholesta-5,7,10(19)-triene-1,3-diol
(5Z,7E)-(1S,3R,24S)-25-(5-butyloxazole-2-yl)-24-[[(ethylamino)carbonyl]oxy]-26,27-cyclo-9,10-secocholesta-5,7,10(19)-triene-1,3-diol
(5Z,7E)-(1S,3R,24R)-25-(5-butyloxazole-2-yl)-24-[[(ethylamino)carbonyl]oxy]-26,27-cyclo-9,10-secocholesta-5,7,10(19)-triene-1,3-diol
(5Z,7E)-(1S,3R,24S)-25-(5-butyloxazole-2-yl)-24-[[(methylamino)carbonyl]oxy]-26,27-cyclo-9,10-secocholesta-5,7,10(19)-triene-1,3-diol
(5Z,7E)-(1S,3R,24R)-25-(5-butyloxazole-2-yl)-24-[[(methylamino)carbonyl]oxy]-26,27-cyclo-9,10-secocholesta-5,7,10(19)-triene-1,3-diol
(5Z,7E)-(1S,3R,24S)-25-(5-butyloxazole-2-yl)-24-[[((pentafluorophenyl)amino)-carbonyl]oxy]-26,27-cyclo-9,10-secocholesta-5,7,10(19)-triene-1,3-diol
(5Z,7E)-(1S,3R,24R)-25-(5-butyloxazole-2-yl)-24-[[((pentafluorophenyl)amino)-carbonyl]oxy]-26,27-cyclo-9,10-secocholesta-5,7,10(19)-triene-1,3-diol
(5Z,7E)-(1S,3R,24S)-25-(5-butyloxazole-2-yl)-24-[[(2,2-dimethyl-3-hydroxy)-1-oxopropyl]oxy]-26,27-cyclo-9,10-secocholesta-5,7,10(19)-triene-1,3-diol
(5Z,7E)-(1S,3R,24R)-25-(5-butyloxazole-2-yl)-24-[[(2,2-dimethyl-3-hydroxy)-1-oxopropyl]oxy]-26,27-cyclo-9,10-secocholesta-5,7,10(19)-triene-1,3-diol
(5Z,7E,22E)-(1S,3R,24S)-25-(5-butyloxazole-2-yl)-26,27-cyclo-9,10-secocholesta-5,7,10(19),22-tetraene-1,3,24-triol tris-isonicotinate
(5Z,7E,22E)-(1S,3R,24R)-25-(5-butyloxazole-2-yl)-26,27-cyclo-9,10-secocholesta-5,7,10(19),22-tetraene-1,3,24-triol tris-isonicotinate
(5Z,7E,22E)-(1S,3R,24S)-25-(5-butyloxazole-2-yl)-26,27-cyclo-9,10-secocholesta-5,7,10(19),22-tetraene-1,3,24-triol tribenzoate
(5Z,7E,22E)-(1S,3R,24R)-25-(5-butyloxazole-2-yl)-26,27-cyclo-9,10-secocholesta-5,7,10(19),22-tetraene-1,3 ,24-triol tribenzoate
(5Z,7E,22E)-(1S,3R,24S)-24-acetoxy-26,27-cyclo-9,10-secocholesta-5,7,10(19),22-tetraene-1,3-diol
(5Z,7E,22E)-(1S,3R,24R)-24-acetoxy-26,27-cyclo-9,10-secocholesta-5,7,10(19),22-tetraene-1,3-diol
(5Z,7E,22E)-(1S,3R,24S)-24-(2,2-dimethyl-1-oxopropoxy)-26,27-cyclo-9,10-secocholesta-5,7,10(19),22-tetraene-1,3-diol
(5Z,7E,22E)-(1S,3R,24R)-24-(2,2-dimethyl-1-oxopropoxy)-26,27-cyclo-9,10-secocholesta-5,7,10(19),22-tetraene-1,3-diol
(5Z,7E,22E)-(1S,3R,24S)-24-(1-oxobutoxy)-26,27-cyclo-9,10-secocholesta-5,7,10(19),22-tetraene-1,3-diol
(5Z,7E,22E)-(1S,3R,24R)-24-(1-oxobutoxy)-26,27-cyclo-9,10-secocholesta-5,7,10(19),22-tetraene-1,3-diol
(5Z,7E,22E)-(1S,3R,24S)-24-(2-methyl-1-oxobutoxy)-26,27-cyclo-9,10-secocholesta-5,7,10(19),22-tetraene-1,3-diol
(5Z,7E,22E)-(1S,3R,24R)-24-(2-methyl-1-oxobutoxy)-26,27-cyclo-9,10-secocholesta-5,7,10(19),22-tetraene-1,3-diol
(5Z,7E,22E)-(1S,3R,24S)-24-(1-oxopropoxy)-26,27-cyclo-9,10-secocholesta-5,7,10(19),22-tetraene-1,3-diol
(5Z,7E,22E)-(1S,3R,24R)-24-(1-oxopropoxy)-26,27-cyclo-9,10-secocholesta-5,7,10(19),22-tetraene-1,3-diol
(5Z,7E,22E)-(1S,3R,24S)-24-(1-oxopentoxy)-26,27-cyclo-9,10-secocholesta-5,7,10(19),22-tetraene-1,3-diol
(5Z,7E,22E)-(1S,3R,24R)-24-(1-oxopentoxy)-26,27-cyclo-9,10-secocholesta-5,7,10(19),22-tetraene-1,3-diol
(5Z,7E,22E)-(1S,3R,24S)-24-benzoyloxy-26,27-cyclo-9,10-secocholesta-5,7,10(19),22-tetraene-1,3-diol
(5Z,7E,22E)-(1S,3R,24R)-24-benzoyloxy-26,27-cyclo-9,10-secocholesta-5,7,10(19),22-tetraene-1,3-diol
(5Z,7E,22E)-(1S,3R,24S)-24-[[(methylamino)carbonyl]oxy]-26,27-cyclo-9,10-secocholesta-5,7,10(19),22-tetraene-1,3-diol
(5Z,7E,22E)-(1S,3R,24R)-24-[[(methylamino)carbonyl]oxy]-26,27-cyclo-9,10-secocholesta-5,7,10(19),22-tetraene-1,3-diol
(5Z,7E,22E)-(1S,3R,24S)-24-[[(ethylamino)carbonyl]oxy]-26,27-cyclo-9,10-secocholesta-5,7,10(19),22-tetraene-1,3-diol
(5Z,7E,22E)-(1S,3R,24R)-24[[(ethylamino)carbonyl]oxy]-26,27-cyclo-9,10-secocholesta-5,7,10(19),22-tetraene-1,3-diol
(5Z,7E,22E)-(1S,3R,24S)-24-(4-pyridylcarbonyl)-26,27-cyclo-9,10-secocholesta-5,7,10(19),22-tetraene-1,3-diol
(5Z,7E,22E)-(1S,3R,24R)-24-(4-pyridylcarbonyl)-26,27-cyclo-9,10-secocholesta-5,7,10(19),22-tetraene-1,3-diol
(5Z,7E,22E)-(1S,3R,24S)-24-(3-hydroxy-2-methyl-1-oxopropoxy)-26,27-cyclo-9,10-secocholesta-5,7,10(19),22-tetraene-1,3-diol
(5Z,7E,22E)-(1S,3R,24R)-24-(3-hydroxy-2-methyl-1-oxopropoxy)-26,27-cyclo-9,10-secocholesta-5,7,10(19),22-tetraene-1,3-diol
(5Z,7E,22E)-(1S,3R,24S)-24-(2-methyl-1-oxopropoxy)-26,27-cyclo-9,10-secocholesta-5,7,10(19),22-tetraene-1,3-diol
(5Z,7E,22E)-(1S,3R,24R)-24-(2-methyl-1-oxopropoxy)-26,27-cyclo-9,10-secocholesta-5,7,10(19),22-tetraene-1,3-diol
(5Z,7E)-(1S,3R,24S)-24-[[((pentafluorophenyl)amino)-carbonyl]oxy]-26,27-cyclo-9,10-secocholesta-5,7,10(19)-triene-1,3-diol
(5Z,7E)-(1S,3R,24R)-24-[[((pentafluorophenyl)amino)-carbonyl]oxy]-26,27-cyclo-9,10-secocholesta-5,7,10(19)-triene-1,3-diol
(5Z,7E)-(1S,3R,24S)-24-[[(2,2-dimethyl-3-hydroxy)-1-oxopropyl]oxy]-26,27-cyclo-9,10-secocholesta-5,7,10(19)-triene-1,3-diol
(5Z,7E)-(1S,3R,24R)-24-[[(2,2-dimethyl-3-hydroxy)-1-oxopropyl]oxy]-26,27-cyclo-9,10-secocholesta-5,7,10(19)-triene-1,3-diol
(5Z,7E,22E)-(1S,3R,24S)-26,27-cyclo-9,10-secocholesta-5,7,10(19),22-tetraene-1,3,24-triol tris-isonicotinate
(5Z,7E,22E)-(1S,3R,24R)-26,27-cyclo-9,10-secocholesta-5,7,10(19),22-tetraene-1,3,24-triol tris-isonicotinate
(5Z,7E,22E)-(1S,3R,24S)-26,27-cyclo-9,10-secocholesta-5,7,10(19),22-tetraene-1,3,24-triol tribenzoate
(5Z,7E,22E)-(1S,3R,24R)-26,27-cyclo-9,10-secocholesta-5,7,10(19),22-tetraene-1,3,24-triol tribenzoate
(5Z,7E)-(1S,3R,24S)-24-acetoxy-25-(5-butylthiazole-2-yl)-26,27-cyclo-9,10-secocholesta-5,7,10(19)-triene-1,3-diol
(5Z,7E)-(1S,3R,24R)-24-acetoxy-25-(5-butylthiazole-2-yl)-26,27-cyclo-9,10-secocholesta-5,7,10(19)-triene-1,3-diol
(5Z,7E)-(1S,3R,24S)-25-(5-butylthiazole-2-yl)-24-(2,2-dimethyl-1-oxopropoxy)-26,27-cyclo-9,10-secocholesta-5,7,10(19)-triene-1,3-diol
(5Z,7E)-(1S,3R,24R)-25-(5-butylthiazole-2-yl)-24-(2,2-dimethyl-1-oxopropoxy)-26,27-cyclo-9,10-secocholesta-5,7,10(19)-triene-1,3-diol
(5Z,7E)-(1S,3R,24S)-25-(5-butylthiazole-2-yl)-24-(1-oxobutoxy)-26,27-cyclo-9,10-secocholesta-5,7,10(19)-triene-1,3-diol
(5Z,7E)-(1S,3R,24R)-25-(5-butylthiazole-2-yl)-24-(1-oxobutoxy)-26,27-cyclo-9,10-secocholesta-5,7,10(19)-triene-1,3-diol
(5Z,7E)-(1S,3R,24S)-25-(5-butylthiazole-2-yl)-24-(2-methyl-1-oxobutoxy)-26,27-cyclo-9,10-secocholesta-5,7,10(19)-triene-1,3-diol
(5Z,7E)-(1S,3R,24R)-25-(5-butylthiazole-2-yl)-24-(2-methyl-1-oxobutoxy)-26,27-cyclo-9,10-secocholesta-5,7,10(19)-triene-1,3-diol
(5Z,7E)-(1S,3R,24S)-25-(5-butylthiazole-2-yl)-24-(1-oxopropoxy)-26,27-cyclo-9,10-secocholesta-5,7,10(19)-triene-1,3-diol
(5Z,7E)-(1S,3R,24R)-25-(5-butylthiazole-2-yl)-24-(1-oxopropoxy)-26,27-cyclo-9,10-secocholesta-5,7,10(19)-triene-1,3-diol
(5Z,7E)-(1S,3R,24S)-25-(5-butylthiazole-2-yl)-24-(1-oxopentoxy)-26,27-cyclo-9,10-secocholesta-5,7,10(19)-triene-1,3-diol
(5Z,7E)-(1S,3R,24R)-25-(5-butylthiazole-2-yl)-24-(1-oxopentoxy)-26,27-cyclo-9,10-secocholesta-5,7,10(19)-triene-1,3-diol
(5Z,7E)-(1S,3R,24S)-24-benzoyloxy-25-(5-butylthiazole-2-yl)-26,27-cyclo-9,10-secocholesta-5,7,10(19)-triene-1,3-diol
(5Z,7E)-(1S,3R,24R)-24-benzoyloxy-25-(5-butylthiazole-2-yl)-26,27-cyclo-9,10-secocholesta-5,7,10(19)-triene-1,3-diol
(5Z,7E)-(1S,3R,24S)-25-(5-butylthiazole-2-yl)-26,27-cyclo-24-(4-pyridylcarbonyl)-9,10-secocholesta-5,7,10(19)-triene-1,3-diol
(5Z,7E)-(1S,3R,24R)-25-(5-butylthiazole-2-yl)-26,27-cyclo-24-(4-pyridylcarbonyl)-9,10-secocholesta-5,7,10(19)-triene-1,3-diol
(5Z,7E)-(1S,3R,24S)-25-(5-butylthiazole-2-yl)-24-[[(ethylamino)carbonyl]oxy]-26,27-cyclo-9,10-secocholesta-5,7,10(19)-triene-1,3-diol
(5Z,7E)-(1S,3R,24R)-25-(5-butylthiazole-2-yl)-24-[[(ethylamino)carbonyl]oxy]-26,27-cyclo-9,10-secocholesta-5,7,10(19)-triene-1,3-diol
(5Z,7E)-(1S,3R,24S)-25-(5-butylthiazole-2-yl)-24-[[(methylamino)carbonyl]oxy]-26,27-cyclo-9,10-secocholesta-5,7,10(19)-triene-1,3-diol
(5Z,7E)-(1S,3R,24R)-25-(5-butylthiazole-2-yl)-24-[[(methylamino)carbonyl]oxy]-26,27-cyclo-9,10-secocholesta-5,7,10(19)-triene-1,3-diol
(5Z,7E)-(1S,3R,24S)-25-(5-butylthiazole-2-yl)-24-(3-hydroxy-2-methyl-1-oxopropoxy)-26,27-cyclo-9,10-secocholesta-5,7,10(19)-triene-1,3-diol
(5Z,7E)-(1S,3R,24R)-25-(5-butylthiazole-2-yl)-24-(3-hydroxy-2-methyl-1-oxopropoxy)-26,27-cyclo-9,10-secocholesta-5,7,10(19)-triene-1,3-diol
(5Z,7E)-(1S,3R,24S)-25-(5-butylthiazole-2-yl)-24-(2-methyl-1-oxopropoxy)-26,27-cyclo-9,10-secocholesta-5,7,10(19)-triene-1,3-diol
(5Z,7E)-(1S,3R,24R)-25-(5-butylthiazole-2-yl)-24-(2-methyl-1-oxopropoxy)-26,27-cyclo-9,10-secocholesta-5,7,10(19)-triene-1,3-diol
(5Z,7E)-(1S,3R,24S)-25-(5-butylthiazole-2-yl)-24-[[((pentafluorophenyl)amino)-carbonyl]oxy]-26,27-cyclo-9,10-secocholesta-5,7,10(19)-triene-1,3-diol
(5Z,7E)-(1S,3R,24R)-25-(5-butylthiazole-2-yl)-24-[[((pentafluorophenyl)amino)-carbonyl]oxy]-26,27-cyclo-9,10-secocholesta-5,7,10(19)-triene-1,3-diol
(5Z,7E)-(1S,3R,24S)-25-(5-butylthiazole-2-yl)-24-[[(2,2-dimethyl-3-hydroxy)-1-oxopropyl]oxy]-26,27-cyclo-9,10-secocholesta-5,7,10(19)-triene-1,3-diol
(5Z,7E)-(1S,3R,24R)-25-(5-butylthiazole-2-yl)-24-[[(2,2-dimethyl-3-hydroxy)-1-oxopropyl]oxy]-26,27-cyclo-9,10-secocholesta-5,7,10(19)-triene-1,3-diol
(5Z,7E,22E)-(1S,3R,24S)-25-(5-butylthiazole-2-yl)-26,27-cyclo-9,10-secocholesta-5,7,10(19),22-tetraene-1,3,24-triol tris-isonicotinate
(5Z,7E,22E)-(1S,3R,24R)-25-(5-butylthiazole-2-yl)-26,27-cyclo-9,10-secocholesta-5,7,10(19),22-tetraene-1,3,24-triol tris-isonicotinate
(5Z,7E,22E)-(1S,3R,24S)-25-(5-butylthiazole-2-yl)-26,27-cyclo-9,10-secocholesta-5,7,10(19),22-tetraene-1,3,24-triol tribenzoate
(5Z,7E,22E)-(1S,3R,24R)-25-(5-butylthiazole-2-yl)-26,27-cyclo-9,10-secocholesta-5,7,10(19),22-tetraene-1,3,24-triol tribenzoate
(5Z,7E)-(1S,3R,24S)-24-acetoxy-25-(4-methylthiazole-2-yl)-26,27-cyclo-9,10-secocholesta-5,7,10(19)-triene-1,3-diol
(5Z,7E)-(1S,3R,24R)-24-acetoxy-25-(-methylthiazole-2-yl)-26,27-cyclo-9,10-secocholesta-5,7,10(19)-triene-1,3-diol
(5Z,7E)-(1S,3R,24S)-24-(2,2-dimethyl-1-oxopropoxy)-25-(4-methylthiazole-2-yl)-26,27-cyclo-9,10-secocholesta-5,7,10(19)-triene-1,3-diol
(5Z,7E)-(1S,3R,24R)-24-(2,2-dimethyl-1-oxopropoxy)-25-(4-methylthiazole-2-yl)-26,27-cyclo-9,10-secocholesta-5,7,10(19)-triene-1,3-diol
(5Z,7E)-(1S,3R,24S)-25-(4-methylthiazole-2-yl)-24-(1-oxobutoxy)-26,27-cyclo-9,10-secocholesta-5,7,10(19)-triene-1,3-diol
(5Z,7E)-(1S,3R,24R)-25-(4-methylthiazole-2-yl)-24-(1-oxobutoxy)-26,27-cyclo-9,10-secocholesta-5,7,10(19)-triene-1,3-diol
(5Z,7E)-(1S,3R,24S)-24-(2-methyl-1-oxobutoxy)-25-(4-methylthiazole-2-yl)-26,27-cyclo-9,10-secocholesta-5,7,10(19)-triene-1,3-diol
(5Z,7E)-(1S,3R,24R)-24-(2-methyl-1-oxobutoxy)-25-(4-methylthiazole-2-yl)-26,27-cyclo-9,10-secocholesta-5,7,10(19)-triene-1,3-diol
(5Z,7E)-(1S,3R,24S)-25-(4-methylthiazole-2-yl)-24-(1-oxopropoxy)-26,27-cyclo-9,10-secocholesta-5,7,10(19)-triene-1,3-diol
(5Z,7E)-(1S,3R,24R)-25-(4-methylthiazole-2-yl)-24-(1-oxopropoxy)-26,27-cyclo-9,10-secocholesta-5,7,10(19)-triene-1,3-diol
(5Z,7E)-(1S,3R,24S)-25-(4-methylthiazole-2-yl)-24-(1-oxopentyloxy)-26,27-cyclo-9,10-secocholesta-5,7,10(19)-triene-1,3-diol
(5Z,7E)-(1S,3R,24R)-25-(4-methylthiazole-2-yl)-24-(1-oxopentoxy)-26,27-cyclo-9,10-secocholesta-5,7,10(19)-triene-1,3-diol
(5Z,7E)-(1S,3R,24S)-24-benzoyloxy-25-(4-methylthiazole-2-yl)-26,27-cyclo-9,10-secocholesta-5,7,10(19)-triene-1,3-diol
(5Z,7E)-(1S,3R,24R)-24-benzoyloxy-25-(4-methylthiazole-2-yl)-26,27-cyclo-9,10-secocholesta-5,7,10(19)-triene-1,3-diol
(5Z,7E)-(1S,3R,24S)-25-(4-methylthiazole-2-yl)-26,27-cyclo-24-(4-pyridylcarbonyl)-9,10-secocholesta-5,7,10(19)-triene-1,3-diol
(5Z,7E)-(1S,3R,24R)-25-(4-methylthiazole-2-yl)-26,27-cyclo-24-(4-pyridylcarbonyl-9,10-secocholesta-5,7,10(19)-triene-1,3-diol
(5Z,7E)-(1S,3R,24S)-24-[[(ethylamino)carbonyl]oxy]-25-(4-methylthiazole-2-yl)-26,27-cyclo-9,10-secocholesta-5,7,10(19)-triene-1,3-diol
(5Z,7E)-(S,3R,24R)-24-[[(ethylamino)carbonyl]oxy]-25-(4-methylthiazole-2-yl)-26,27-cyclo-9,10-secocholesta-5,7,10(19)-triene-1,3-diol
(5Z,7E)-(1S,3R,24S)-24-[[(methylamino)carbonyl]oxy]-25-(4-methylthiazole-2-yl)-26,27-cyclo-9,10-secocholesta-5,7,10(19)-triene-1,3-diol
(5Z,7E)-(1S,3R,24R)-24-[[(methylamino)carbonyl]oxy]-25-(4-methylthiazole-2-yl)-26,27-cyclo-9,10-secocholesta-5,7,10(19)-triene-1,3-diol
(5Z,7E)-(1S,3R,24S)-24-(3-hydroxy-2-methyl-1-oxopropoxy)-25-(4-methyl-thiazole-2-yl)-26,27-cyclo-9,10-secocholesta-5,7,10(19)-triene-1,3-diol
(5Z,7E)-(1S,3R,24R)-24-(3-hydroxy-2-methyl-1-oxopropoxy)-25-(4-methyl-thiazole-2-yl)-26,27-cyclo-9,10-secocholesta-5,7,10(19)-triene-1,3-diol
(5Z,7E)-(1S,3R,24S)-24-(2-methyl-1-oxopropoxy)-25-(4-methylthiazole-2-yl)-26,27-cyclo-9,10-secocholesta-5,7,10(19)-triene-1,3-diol
(5Z,7E)-(1S,3R,24R)-24-(2-methyl-1-oxopropoxy)-25-(4-methylthiazole-2-yl)-26,27cyclo-9,10-secocholesta-5,7,10(19)-triene-1,3-diol
(5Z,7E)-(1S,3R,24S)-25-(4-methylthiazole-2-yl)-(24-[[((pentafluorophenyl)amino)-carbonyl]oxy]-26,27-cyclo-9,10-secocholesta-5,7,10(19)-triene-1,3-diol
(5Z,7E)-(1S,3R,24R)-25-(4-methylthiazole-2-yl)-24-[[((pentafluorophenyl)amino)-carbonyl]oxy]-26,27-cyclo-9,10-secocholesta-5,7,10(19)-triene-1,3-diol
(5Z,7E)-(1S,3R,24S)-24-[[(2,2-dimethyl-3-hydroxy)-1-oxopropyl]oxy]-25-(4-methylthiazole-2-yl)-26,27-cyclo-9,10-secocholesta-5,7,10(19)-triene-1,3-diol
(5Z,7E)-(1S,3R,24R)-24-[[(2,2-dimethyl-3-hydroxy)-1-oxopropyl]oxy]-25-(4-methylthiazole-2-yl)-26,27-cyclo-9,10-secocholesta-5,7,10(19)-triene-1,3-diol
(5Z,7E,22E)-(1S,3R,24S)-25-(4-methylthiazole-2-yl)-26,27-cyclo-9,10-secocholesta-5,7,10(19),22-tetraene-1,3,24-triol tris-isonicotinate
(5Z,7E,22E)-(1S,3R,24R)-25-(4-methylthiazole-2-yl)-26,27-cyclo-9,10-secocholesta-5,7,10(19),22-tetraene-1,3,24-triol tris-isonicotinate
(5Z,7E,22E)-(1S,3R,24S)-25-(4-methylthiazole-2-yl)-26,27-cyclo-9,10-secocholesta-5,7,10(19),22-tetraene-1,3,24-triol tribenzoate
(5Z,7E,22E)-(1S,3R,24R)-25-(4-methylthiazole-2-yl)-26,27-cyclo-9,10-secocholesta-5,7,10(19),22-tetraene-1,3,24-triol tribenzoate
(5Z,7E)-(1S,3R,24S)-24-acetoxy-25-(4-methyloxazole-2-yl)-26,27-cyclo-9,10-secocholesta-5,7,10(19)-triene-1,3-diol
(5Z,7E)-(1S,3R,24R)-24-acetoxy-25-(4-methyloxazole-2-yl)-26,27-cyclo-9,10-secocholesta-5,7,10(19)-triene-1,3-diol
(5Z,7E)-(1S,3R,24S)-24-(2,2-dimethyl-1-oxopropoxy)-25-(4-methyloxazole-2-yl)-26,27-cyclo-9,10-secocholesta-5,7,10(19)-triene-1,3-diol
(5Z,7E)-(1S,3R,24R)-24-(2,2-dimethyl-1-oxopropoxy)-25-(4-methyloxazole-2-yl)-26,27-cyclo-9,10-secocholesta-5,7,10(19)-triene-1,3-diol
(5Z,7E)-(1S,3R,24S)-25-(4-methyloxazole-2-yl)-24-(1-oxobutoxy)-26,27-cyclo-9,10-secocholesta-5,7,10(19)-triene-1,3-diol
(5Z,7E)-(1S,3R,24R)-25-(4-methyloxazole-2-yl)-24-(1-oxobutoxy)-26,27-cyclo-9,10-secocholesta-5,7,10(19)-triene-1,3-diol
(5Z,7E)-(1S,3R,24S)-25-(4-methyloxazole-2-yl)-24-(2-methyl-1-oxobutoxy)-26,27-cyclo-9,10-secocholesta-5,7,10(19)-triene-1,3-diol
(5Z,7E)-(1S,3R,24R)-25-(4-methyloxazole-2-yl)-24-(2-methyl-1-oxobutoxy)-26,27-cyclo-9,10-secocholesta-5,7,10(19)-triene-1,3-diol
(5Z,7E)-(1S,3R,24S)-25-(4-methyloxazole-2-yl)-24-(1-oxopropoxy)-26,27-cyclo-9,10-secocholesta-5,7,10(19)-triene-1,3-diol
(5Z,7E)-(1S,3R,24R)-25-(4-methyloxazole-2-yl)-24-(1-oxopropoxy)-26,27-cyclo-9,10-secocholesta-5,7,10(19)-triene-1,3-diol
(5Z,7E)-(1S,3R,24S)-25-(4-methyloxazole-2-yl)-24-(1-oxopentoxy)-26,27-cyclo-9,10-secocholesta-5,7,10(19)-triene-1,3-diol
(5Z,7E)-(1S,3R,24R)-25-(4-methyloxazole-2-yl)-24-(1-oxopentoxy)-26,27-cyclo-9,10-secocholesta-5,7,10(19)-triene-1,3-diol
(5Z,7E)-(1S,3R,24S)-24-benzoyloxy-25-(4-methyloxazole-2-yl)-26,27-cyclo-9,10-secocholesta-5,7,10(19)-triene-1,3-diol
(5Z,7E)-(1S,3R,24R)-24-benzoyloxy-25-(4-methyloxazole-2-yl)-26,27-cyclo-9,10-secocholesta-5,7,10(19)-triene-1,3-diol
(5Z,7E)-(1S,3R,24S)-25-(4-methyloxazole-2-yl)-26,27-cyclo-24-(4-pyridylcarbonyl)-9,10-secocholesta-5,7,10(19)-triene-1,3-diol
(5Z,7E)-(1S,3R,24R)-25-(4-methyloxazole-2-yl)-26,27-cyclo-24-(4-pyridylcarbonyl)-9,10-secocholesta-5,7,10(19)-triene-1,3-diol
(5Z,7E)-(1S,3R,24S)-24-[[(ethylamino)carbonyl]oxy]-25-(4-methyloxazole-2-yl)-26,27-cyclo-9,10-secocholesta-5,7,10(19)-triene-1,3-diol
(5Z,7E)-(1S,3R,24R)-24-[[(ethylamino)carbonyl]oxy]-25-(4-methyloxazole-2-yl)-26,27-cyclo-9,10-secocholesta-5,7,10(19)-triene-1,3-diol
(5Z,7E)-(1S,3R,24S)-24-[[(methylamino)carbonyl]oxy]-25-(4-methyloxazole-2-yl)-26,27-cyclo-9,10-secocholesta-5,7,10(19)-triene-1,3-diol
(5Z,7E)-(1S,3R,24R)-24-[[(methylamino)carbonyl]oxy]-25-(4-methyloxazole-2-yl)-26,27-cyclo-9,10-secocholesta-5,7,10(19)-triene-1,3-diol
(5Z,7E)-(1S,3R,24S)-24-(3-hydroxy-2-methyl-1-oxopropoxy)-25-(4-methyl-oxazole-2-yl)-26,27-cyclo-9,10-secocholesta-5,7,10(19)-triene-1,3-diol
(5Z,7E)-(1S,3R,24R)-24-(3-hydroxy-2-methyl-1-oxopropoxy)-25-(4-methyl-oxazole-2-yl)-26,27-cyclo-9,10-secocholesta-5,7,10(19)-triene-1,3-diol
(5Z,7E)-(1S,3R,24S)-25-(4-methyloxazole-2-yl)-24-(2-methyl-1-oxopropoxy)-26,27-cyclo-9,10-secocholesta-5,7,10(19)-triene-1,3-diol
(5Z,7E)-(1S,3R,24R)-25-(4-methyloxazole-2-yl)-24-(2-methyl-1-oxopropoxy)-26,27-cyclo-9,10-secocholesta-5,7,10(19)-triene-1,3-diol
(5Z,7E)-(1S,3R,24S)-25-(4-methyloxazole-2-yl)-(24-[[((pentafluorophenyl)amino)-carbonyl]oxy]-26,27-cyclo-9,10-secocholesta-5,7,10(19)-triene-1,3-diol
(5Z,7E)-(1S,3R,24R)-25-(4-methyloxazole-2-yl)-24-[[((pentafluorophenyl)amino)-carbonyl]oxy]-26,27-cyclo-9,10-secocholesta-5,10(19)-triene-1,3-diol
(5Z,7E)-(1S,3R,24S)-24-[[(2,2-dimethyl-3-hydroxy)-1-oxopropyl]oxy]-25-(4-methyloxazole-2-yl)-26,27-cyclo-9,10-secocholesta-5,7,10(19)-triene-1,3-diol
(5Z,7E)-(1S,3R,24R)-24-[[(2,2-dimethyl-3-hydroxy)-1-oxopropyl]oxy]-25-(4-methyloxazole-2-yl)-26,27-cyclo-9,10-secocholesta-5,7,10(19)-triene-1,3-diol
(5Z,7E,22E)-(1S,3R,24S)-25-(4-methyloxazole-2-yl)-26,27-cyclo-9,10-secocholesta-5,7,10(19),22-tetraene-1,3,24-triol tris-isonicotinate
(5Z,7E,22E)-(1S,3R,24R)-25-(4-methyloxazole-2-yl)-26,27-cyclo-9,10-secocholesta-5,7,10(19),22-tetraene-1,3,24-triol tris-isonicotinate
(5Z,7E,22E)-(1S,3R,24S)-25-(4-methyloxazole-2-yl)-26,27-cyclo-9,10-secocholesta-5,7,10(19),22-tetraene-1,3,24-triol tribenzoate
(5Z,7E,22E)-(1S,3R,24R)-25-(4-methyloxazole-2-yl)-26,27-cyclo-9,10-secocholesta-5,7,10(19),22-tetraene-1,3,24-triol tribenzoate
(5Z,7E)-(1S,3R,24S)-24-acetoxy-26,27-cyclo-25-(3-propyl-1,2,4-oxadiazole-5-yl)-26,27-cyclo-9,10-secocholesta-5,7,10(19)-triene-1,3-diol
(5Z,7E)-(1S,3R,24R)-24-acetoxy-26,27-cyclo-25-(3-propyl-1,2,4-oxadiazole-5-yl)-26,27-cyclo-9,10-secocholesta-5,7,10(19)-triene-1,3-diol
(5Z,7E)-(1S,3R,24S)-24-(2,2-dimethyl-1-oxopropoxy)-25-(3-propyl-1,2,4-oxadiazole-5-yl)-26,27-cyclo-9,10-secocholesta-5,7,10(19)-triene-1,3-diol
(5Z,7E)-(1S,3R,24R)-24-(2,2-dimethyl-1-oxopropoxy)-25-(3-propyl-1,2,4-oxadiazole-5-yl)-26,27-cyclo-9,10-secocholesta-5,7,10(19)-triene-1,3-diol
(5Z,7E)-(1S,3R,24S)-24-(1-oxobutoxy)-25-(3-propyl-1,2,4-oxadiazole-5-yl)-26,27-cyclo-9,10-secocholesta-5,7,10(19)-triene-1,3-diol
(5Z,7E)-(1S,3R,24R)-24-(1-oxobutoxy)-25-(3-propyl-1,2,4-oxadiazole-5-yl)-26,27-cyclo-9,10-secocholesta-5,7,10(19)-triene-1,3-diol
(5Z,7E)-(1S,3R,24S)-24-(2-methyl-1-oxobutoxy)-25-(3-propyl-1,2,4-oxadiazole-5-yl)-26,27-cyclo-9,10-secocholesta-5,7,10(19)-triene-1,3-diol
(5Z,7E)-(1S,3R,24R)-24-(2-methyl-1-oxobutoxy)-25-(3-propyl-1,2,4-oxadiazole-5-yl)-26,27-cyclo-9,10-secocholesta-5,7,10(19)-triene-1,3-diol
(5Z,7E)-(1S,3R,24S)-24-(1-oxopropoxy)-25-(3-propyl-1,2,4-oxadiazole-5-yl)-26,27-cyclo-9,10-secocholesta-5,7,10(19)-triene-1,3-diol
(5Z,7E)-(1S,3R,24R)-24-(1-oxopropoxy)-25-(3-propyl-1,2,4-oxadiazole-5-yl)-26,27-cyclo-9,10-secocholesta-5,7,10(19)-triene-1,3-diol
(5Z,7E)-(1S,3R,24S)-24-(1-oxopentoxy)-25-(3-propyl-1,2,4-oxadiazole-5-yl)-26,27-cyclo-9,10-secocholesta-5,7,10(19)-triene-1,3-diol
(5Z,7E)-(1S,3R,24R)-24-(1-oxopentoxy)-25-(3-propyl-1,2,4-oxadiazole-5-yl)-26,27-cyclo-9,10-secocholesta-5,7,10(19)-triene-1,3-diol
(5Z,7E)-(1S,3R,24S)-24-benzoyloxy-25-(3-propyl-1,2,4-oxadiazole-5-yl)-26,27-9,10-secocholesta-5,7,10(19)-triene-1,3-diol
(5Z,7E)-(1S,3R,24R)-24-benzoyloxy-25-(3-propyl-1,2,4-oxadiazole-5-yl)-26,27-9,10-secocholesta-5,7,10(19)-triene-1,3-diol
(5Z,7E)-(1S,3R,24S)-25-(3-propyl-1,2,4-oxadiazole-5-yl)-24-(4-pyridylcarbonyl)-26,27-cyclo-9,10-secocholesta-5,7,10(19)-triene-1,3-diol
(5Z,7E)-(1S,3R,24R)-25-(3-propyl-1,2,4-oxadiazole-5-yl)-24-(4-pyridylcarbonyl)-26,27-cyclo-9,10-secocholesta-5,7,10(19)-triene-1,3-diol
(5Z,7E)-(1S,3R,24S)-24-[[(ethylamino)carbonyl]oxy]-25-(3-propyl-1,2,4-pyridylcarbonyl)-26,27-cyclo-9,10-secocholesta-5,7,10(19)-triene-1,3-diol
(5Z,7E)-(1S,3R,24R)-24-[[(ethylamino)carbonyl]oxy]-25-(3-propyl-1,2,4-pyridylcarbonyl)-26,27-cyclo-9,10-secocholesta-5,7,10(19)-triene-1,3-diol
(5Z,7E)-(1S,3R,24S)-24-[[(methylamino)carbonyl]oxy]-25-(3-propyl-1,2,4-oxadiazole-5-yl)-26,27-cyclo-9,10-secocholesta-5,7,10(19)-triene-1,3-diol
(5Z,7E)-(1S,3R,24R)-24-[[(methylamino)carbonyl]oxy]-25-(3-propyl-1,2,4-oxadiazole-5yl)-26,27-cyclo-9,10-secocholesta-5,7,10(19)-triene-1,3-diol
(5Z,7E)-(1S,3R,24S)-24-(3-hydroxy-2-methyl-1-oxopropoxy)-25-(3-propyl-1,2,4-oxadiazole-5-yl)-26,27-cyclo-9,10-secocholesta-5,7,10(19)-triene-1,3-diol
(5Z,7E)-(1S,3R,24R)-24-(3-hydroxy-2-methyl-1-oxopropoxy)-25-(3-propyl-1,2,4-oxadiazole-5-yl)-26,27-cyclo-9,10-secocholesta-5,7,10(19)-triene-1,3-diol
(5Z,7E)-(1S,3R,24S)-24-(2-methyl-1-oxopropoxy)-25-(3-propyl-1,2,4-oxadiazole-5-yl)-26,27-cyclo-9,10-secocholesta-5,7,10(19)-triene-1,3-diol
(5Z,7E)-(1S,3R,24R)-24-(2-methyl-1-oxopropoxy)-25-(3-propyl-1,2,4-oxadiazole-5-yl)-26,27-cyclo-9,10-secocholesta-5,7,10(19)-triene-1,3-diol
(5Z,7E)-(1S,3R,24S)-24-[[((pentafluorophenyl)amino)-carbonyl]oxy]-25-(3-propyl-1,2,4-oxadiazole-5-yl)-26,27-cyclo-9,10-secocholesta-5,7,10(19)-triene-1,3-diol
(5Z,7E)-(1S,3R,24R)-)-24-[[((pentafluorophenyl)amino)-carbonyl]oxy]-25-(3-propyl-1,2,4-oxadiazole-5-yl)-26,27-cyclo-9,10-secocholesta-5,7,10(19)-triene-1,3-diol
(5Z,7E)-(1S,3R,24S)-24-[[(2,2-dimethyl-3-hydroxy)-1-oxopropyl]oxy]-25-(1,2,4-oxadiazole-5-yl)-26,27-cyclo-9,10-secocholesta-5,7,10(19)-triene-1,3-diol
(5Z,7E)-(1S,3R,24R)-24-[[(2,2-dimethyl-3-hydroxy)-1-oxopropyl]oxy]-25-(1,2,4-oxadiazole-5-yl)-26,27-cyclo-9,10-secocholesta-5,7,10(19)-triene-1,3-diol
(5Z,7E,22E)-(1S,3R,24S)-25-(3-propyl-1,2,4-oxadiazole-5-yl)-26,27-cyclo-9,10-secocholesta-5,7,10(19),22-tetraene-1,3,24-triol tris-isonicotinate
(5Z,7E,22E)-(1S,3R,24R)-25-(3-propyl-1,2,4-oxadiazole-5-yl)-26,27-cyclo-9,10-secocholesta-5,7,10(19),22-tetraene-1,3,24-triol tris-isonicotinate
(5Z,7E,22E)-(1S,3R,24S)-25-(3-propyl-1,2,4-oxadiazole-5-yl)-26,27-cyclo-9,10-secocholesta-5,7,10(19),22-tetraene-1,3,24-triol tribenzoate
(5Z,7E,22E)-(1S,3R,24R)-25-(3-propyl-1,2,4-oxadiazole-5-yl)-26,27-cyclo-9,10-secocholesta-5,7,10(19),22-tetraene-1,3,24-triol tribenzoate
(5Z,7E)-(1S,3R,24S)-24-acetoxy-25-(4-propylimidazole-2-yl)-26,27-cyclo-9,10-secocholesta-5,7,10(19)-triene-1,3-diol
(5Z,7E)-(1S,3R,24R)-24-acetoxy-25-(4-propylimidazole-2-yl)-26,27-cyclo-9,10-secocholesta-5,7,10(19)-triene-1,3-diol
(5Z,7E)-(1S,3R,24S)-24-(2,2-dimethyl-1-oxopropoxy)-25-(4-propylimidazole-2-yl)-26,27-cyclo-9,10-secocholesta-5,7,10(19)-triene-1,3-diol
(5Z,7E)-(1S,3R,24R)-24-(2,2-dimethyl-1-oxopropoxy)-25-(4-propylamidazole-2-yl)-26,27-cyclo-9,10-secocholesta-5,7,10(19)-triene-1,3-diol
(5Z,7E)-(1S,3R,24S)-24-(1-oxobutoxy)-25-(4-propylimidazole-2-yl)-26,27-cyclo-9,10-secocholesta-5,7,10(19)-triene-1,3-diol
(5Z,7E)-(1S,3R,24R)-24-(1-oxobutoxy)-25-(4-propylimidazole-2-yl)-26,27-cyclo-9,10-secocholesta-5,7,10(19)-triene-1,3-diol
(5Z,7E)-(1S,3R,24S)-24-(2-methyl-1-oxobutoxy)-25-(4-propylimidazole-2-yl)-cyclo-9,10-secocholesta-5,7,10(19)-triene-1,3-diol
(5Z,7E)-(1S,3R,24R)-24-(2-methyl-1-oxobutoxy)-25-(4-propylimidazole-2-yl)-26,27-cyclo-9,10-secocholesta-5,7,10(19)-triene-1,3-diol
(5Z,7E)-(1S,3R,24S)-24-(1-oxopropoxy)-25-(4-propylimidazole-2-yl)-26,27-cyclo-9,10-secocholesta-5,7,10(19)-triene-1,3-diol
(5Z,7E)-(1S,3R,24R)-24-(1-oxopropoxy)-25-(4-propylamidazole-2-yl)-26,27-cyclo-9,10-secocholesta-5,7,10(19)-triene-1,3-diol
(5Z,7E)-(1S,3R,24S)-24-(1-oxopentoxy)-25-(4-propylimidazole-2-yl)-26,27-cyclo-9,10-secocholesta-5,7,10(19)-triene-1,3-diol
(5Z,7E)-(1S,3R,24R)-24-(1-oxopentoxy)-25-(4-propylimidazole-2-yl)-26,27-cyclo-9,10-secocholesta-5,7,10(19)-triene-1,3-diol
(5Z,7E)-(1S,3R,24S)-24-benzoyloxy-25-(4-propylimidazole-2-yl)-26,27-cyclo-9,10-secocholesta-5,7,10(19)-triene-1,3-diol
(5Z,7E)-(1S,3R,24R)-24-benzoyloxy-25-(4-propylimidazole-2-yl)-26,27-cyclo-9,10-secocholesta-5,7,10(19)-triene-1,3-diol
(5Z,7E)-(1S,3R,24S)-25-(4-propylimidazole-2-yl)-26,27-cyclo-24-(4-pyridylcarbonyl)-9,10-secocholesta-5,7,10(19)-triene-1,3-diol
(5Z,7E)-(1S,3R,24R)-25-(4-propylimidazole-2-yl)-26,27-cyclo-24-(4-pyridylcarbonyl)-9,10-secocholesta-5,7,10(19)-triene-1,3-diol
(5Z,7E)-(1S,3R,24S)-24-[[(ethylamino)carbonyl]oxy]-25-(4-propylimidazole-2-y)-26,27-cyclo-9,10-secocholesta-5,7,10(19)-triene-1,3-diol
(5Z,7E)-(1S,3R,24R)-24-[[(ethylamino)carbonyl]oxy]-25-(4-propylimidazole-2-yl)-26,27-cyclo-9,10-secocholesta-5,7,10(19)-triene-1,3-diol
(5Z,7E)-(1S,3R,24S)-24-[[(methylamino)carbonyl]oxy]-25-(4-propylimidazole-2-yl)-26,27-cyclo-9,10-secocholesta-5,7,10(19)-triene-1,3-diol
(5Z,7E)-(1S,3R,24R)-24-[[(methylamino)carbonyl]oxy]-25-(4-propylimidazole-2-yl)-26,27-cyclo-9,10-secocholesta-5,7,10(19)-triene-1,3-diol
(5Z,7E)-(1S,3R,24S)-24-(3-hydroxy-2-methyl-1-oxopropoxy)-25-(4-propylimidazole-2-yl)-26,27-cyclo-9,10-secocholesta-5,7,10(19)-triene-1,3-diol
(5Z,7E)-(1S,3R,24R)-24-(3-hydroxy-2-methyl-1-oxopropoxy)-25-(4-propylimidazole-2-yl)-26,27-cyclo-9,10-secocholesta-5,7,10(19)-triene-1,3-diol
(5Z,7E)-(1S,3R,24S)-24-(2-methyl-1-oxopropoxy)-25-(4-propylimidazole-2-yl)-26,27-cyclo-9,10-secocholesta-5,7,10(19)-triene-1,3-diol
(5Z,7E)-(1S,3R,24R)-24-(2-methyl-1-oxopropoxy)-25-(4-propylamidazole-2-yl)-26,17-cyclo-9,10-secocholesta-5,7,10(19)-triene-1,3-diol
(5Z,7E)-(1S,3R,24S)-24-[[((pentafluorophenyl)amino)-carbonyl]oxy]-25-(4-propylimidazole-2-yl)-26,27-cyclo-9,10-secocholesta-5,7,10(19)-triene-1,3-diol
(5Z,7E)-(1S,3R,24R)-}-24-[[((pentafluorophenyl)amino)-carbonyl]oxy]-25-(4-propylimidazole-2-yl)-26,27-cyclo-9,10-secocholesta-5,7,10(19)-triene-1,3-diol
(5Z,7E)-(1S,3R,24S)-24-[[(2,2-dimethyl-3-hydroxy)-1-oxopropyl]oxy]-25-(4-propylimidazole-2-yl)-26,27-cyclo-9,10-secocholesta-5,7,10(19)-triene-1,3-diol
(5Z,7E)-(1S,3R,24R)-24-[[(2,2-dimethyl-3-hydroxy)-1-oxopropyl]oxy]-25-(4-propylimidazole-2-yl)-26,27-cyclo-9,10-secocholesta-5,7,10(19)-triene-1,3-diol
(5Z,7E,22E)-(1S,3R,24S)-25-(4-propylimidazole-2-yl)-26,27-cyclo-9,10-secocholesta-5,7,10(19),22-tetraene-1,3,24-triol tris-isonicotinate
(5Z,7E,22E)-(1S,3R,24R)-25-(4-propylimidazole-2-yl)-26,27-cyclo-9,10-secocholesta-5,7,10(19),22-tetraene-1,3,24-triol tris-isonicotinate
(5Z,7E,22E)-(1S,3R,24S)-25-(4-propylimidazole-2-yl)-26,27-cyclo-9,10-secocholesta-5,7,10(19),22-tetraene-1,3,24-triol tribenzoate
(5Z,7E,22E)-(1S,3R,24R)-25-(4-propylimidazole-2-yl)-26,27-cyclo-9,10-secocholesta-5,7,10(19),22-tetraene-1,3,24-triol tribenzoate

6. Process for the production of the compounds of general formula I according to claim 1, wherein compounds of general formula III 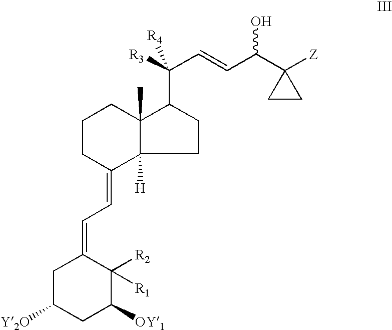
are reacted with compounds of general formula IV or V
Hal-A (IV)
(A)₂O (V)
whereby Hal means chlorine or bromine, in a basic medium to form compounds of general formula 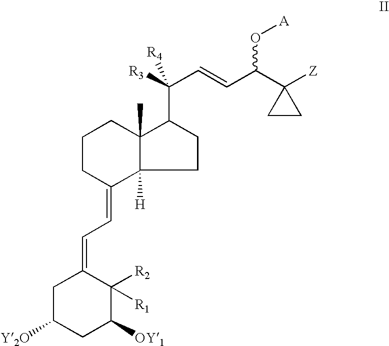
and then the compound of general formula II is converted into a compound of general formula (I) in any sequence by simultaneous or successive cleavage of hydroxy protective groups and optionally by partial, successive or complete esterification of the free hydroxyl groups, and, if desired, the diastereomers are separated in one of the above-mentioned stages.

7. Pharmaceutical preparations that contain at least one compound according to claim 1 together with a pharmaceutically compatible vehicle.

8. Use of the vitamin D derivatives of general formula I for the production of pharmaceutical agents.

9. Use according to claim 8 for the production of pharmaceutical agents for systemic administrations.

10. Use of the vitamin D derivatives of general formula I according to claim 8 for the production of a pharmaceutical agent for the treatment of hyperproliferative diseases of the skin (psoriasis, acne, ichthyosis) as well as tumor diseases and precancerous stages (e.g., tumors of the intestines, carcinomas of the breast, lung tumors, prostate carcinomas, leukemias, T-cell lymphomas, actinic keratoses, cervix dysplasias, in addition to auto-immune diseases (multiple sclerosis, diabetes mellitus type I, myasthenia gravis, lupus erythematosus), rejection reactions in the case of autologous, allogeneic or xenogeneic transplants, as well as AIDS; in addition, the therapeutic use is possible in the case of atrophic skin or wound healing, as well as the therapy of secondary hyperparathyroidism, renal osteodystrophia as well as senile and postmenopausal osteoporosis, diabetes mellitus type II and the therapy of degenerative diseases of the peripheral and central nervous system (Alzheimer's disease and amyotrophic lateral sclerosis) as well as also the regulation of hair growth.

11. Use of vitamin D derivatives of general formula I according to claim 8, which antagonize the action of calcitriol in HL 60 cells, for therapy of hypercalcemias (hypervitaminosis D, intoxication with calcitriol or its analogs), or granulomatous diseases (sarcoidosis, tuberculosis) as well as paraneoplastic hypercalcemias (thus osteolytic metastases and tumors with increased synthesis of parathormone-related peptides) and hypercalcemias in the case of hyperparathyroidism, and for birth control or as immunostimulants as well as in the case of hirsutism and for therapy and prophylaxis of arteriosclerosis, and in addition for therapy of inflammatory diseases (rheumatoid arthritis, Crohn's disease, ulcerative colitis, and granulomatous diseases).
